(19) United States
(12) Patent Application Publication
(10) Pub. No.: US 2017/0333901 A1
(43) Pub. Date: Nov. 23, 2017

(54) CELL-TRAPPING DEVICE, APPARATUS COMPRISING IT AND THEIR USE FOR MICROINJECTION INTO CELLS

(71) Applicant: City University of Hong Kong, Kowloon (HK)

(72) Inventors: Dong Sun, New Territories (HK); Yu Ting Chow, New Territories (HK); Shuxun Chen, Kowloon (HK); Ronald Adolphus Li, Kowloon (HK)

(21) Appl. No.: 15/156,581

(22) Filed: May 17, 2016

(51) Int. Cl. B01L 3/00; C12Q 3/00; C12N 5/077

(52) U.S. Cl. CPC B01L 3/502761; B01L 3/502707; C12N 5/0656; C12Q 3/00; B01L 2200/0668; B01L 2200/025; B01L 2200/12; B01L 2300/0627; B01L 2300/12; B01L 2300/06

(57) ABSTRACT

A cell-trapping device includes a microchannel portion for trapping a plurality of cells with an average diameter of at most 25 μm for high-throughput microinjection of an injectant into the cells. The cell-trapping device includes a microchannel portion having formed therein a cell-trapping area including a plurality of cell-trapping microchannels configured to trap one cell per cell-trapping channel. A method for preparing the cell-trapping device and an apparatus for high-throughput microinjection is also provided. Further provided is a method for injecting an injectant into a plurality of cells. The cell-trapping device, apparatus, and method allow for a rapid and highly reproducible microinjection into small cells with high productivity, high accuracy and a good cell survival rate.

### TECHNICAL FIELD

The present invention provides a cell-trapping device comprising a microchannel portion with cell-trapping microchannels for trapping a plurality of cells with an average diameter of at most 25 μm for high-throughput microinjection of an injectant into the cells. Preferably but not exclusively, the cell-trapping device is for trapping a plurality of human cells with an average diameter of less than 25 μm for high-throughput microinjection of an injectant selected from at least one of polypeptides, proteins, RNA and/or DNA into the cells. The present invention also refers to a method of preparing the cell-trapping device and an apparatus for high-throughput microinjection. The present invention also provides a method for microinjection.

### BACKGROUND OF THE INVENTION

Microinjection is an area of growing technological and commercial interest in which it is desired to cultivate, study, grow and/or modify biological material, in particular cells, using methods that involve injecting extracellular materials into these biological materials.

Microinjection has been widely used in biomedical studies, especially in delivering exogenous macromolecules like DNA and RNA into individual cells. This technique has the advantage of delivering a high concentration of macromolecules into a particular area within the cell. The technique has been used to investigate cellular senescence in human fibroblasts by injecting antibodies, as well as the role of a specific enzyme in the functional maturation of mitotic centrosomes on HeLa cells and Hs68 cells through injecting antibodies. Recent studies on heart regenerative medicine have also demonstrated that microinjection of synthetic modified mRNA can direct the differentiation of heart progenitor cells into cardiovascular cell types.

Most of the existing microinjection processes are conducted manually using microscopic manipulators. During these processes, the operator must simultaneously control the micromanipulator, microinjector, and microscope stage, which is time consuming. Thus, a great demand exists for the development of a robot-aided, i.e. automated microinjection apparatus to accomplish this complex task efficiently and automatically.

Further, research over the past decade has mainly focused on injections in relatively large-scaled biological materials such as cells with a size of 50 μm to 1 mm. Examples include xenopus oocyte (about 1 mm), zebrafish oocyte (about 500 μm), and mouse embryo (about 55 μm). However, only limited research has been conducted on microinjection into cells smaller than 25 μm, which is the typical size of many human cells.

The automated injection of small cells, especially small adherent cells, has several key challenges. Firstly, adherent cells are irregularly shaped, which causes much difficulty to automate the cell recognition process robustly, especially when the cells are very small. Secondly, the height of the cell adhering to the substrate is only a few micrometers, which requires high precision in manipulating the needle and the cell simultaneously. Third, the adherent cell has a much smaller size (below 25 μm) than oocytes (50 to 500 μm).

Several semiautomated adherent cell injection systems have been reported in the literature, most of them with limited and relatively low throughput, limited success rate and/or limited cell survival rate (Matsuoka, H. et al., J. Biotechnol., 2005, 116, 185-194, Lim, S. et al., Pertanika J. Sci. Technol., 2011, 19, 273-283, Viigipuu, K. and Kallio, P., Alternatives Laboratory Animals, 2004, 32, 417-423, Wang, W. et al., Rev. Sci. Instrum., 2008, 79, 104302-1-104302-6). All these systems have not been facilitated with cell recognition capability for automation, either. An automated adherent cell microinjection system has recently been developed (Becattini, G. et al., IEEE J. Biomed. Health Informat., 2014, 18, 83-93), but the throughput and speed that can be obtained are limited and relatively low.

Thus, there remains a strong need due to recent demands arising in the field of medical applications for means and methods for microinjection which overcome at least one of the limitations of the prior art, i.e. which are capable of automation, provide robustness, high-throughput, and/or good reproducibility. In particular means and methods are required with good efficiency, which are suitable for microinjection into a high amount of small cells including adherent cells with diameters of 25 μm and below.

### SUMMARY OF THE INVENTION

In a first aspect of the present invention, a cell-trapping device for trapping a plurality of cells with an average diameter of at most 25 μm for high-throughput microinjection of an injectant into the cells is provided.

The cell-trapping device of the present invention comprises a microchannel portion having formed therein a cell-trapping area. The cell-trapping area comprises a plurality of cell-trapping microchannels configured to trap one cell per cell-trapping microchannel.

The term “microchannel” as used herein refers to channels directing a fluid flow through the cell-trapping device and formed by recesses in one or more materials forming the microchannel portion with a height and width as dimensions perpendicular to the main fluid flow direction through the channels being in the micrometer range, usually of less than 100 μm, further preferably of less than 50 μm and in particular of less than 30 μm. The length of the channels, which is the dimension of the channels in the direction of the main fluid flow though the channels, can be in the micrometer up to the millimeter range.

It should be noted that the terms “channel” and “microchannel” are used interchangeably in this patent application.

The cell-trapping device of the present invention is, thus, a microfluidic chip. The term “microfluidic” is generally known to refer to the use of microchannels for transport of fluids. Typically, microfluidic systems can be designed to handle fluid volumes ranging from the picoliter to the milliliter range.

Microinjection is generally known to a person skilled in the art as a cellular manipulation technique that enables introduction of minute amounts of materials like extracellular materials into biological materials such as cells through insertion of one or more injection needles.

The microinjection of the present invention is a high-throughput microinjection, which is used to refer to a microinjection with a throughput, i.e. injection into more than 10 cells/min, preferably of more than 20 cells/min, further preferably of more than 30 cells/min and in particular of at least about 35 cells/min.

In an embodiment, the high-throughput microinjection is an automated high-throughput microinjection, i.e. with an automated injection process, in particular an automated identification, alignment of the injection needle and injection into trapped cells.

Cell-trapping microchannels are microchannels suitable to trap one cell each, i.e. to immobilize the cell for the microinjection, and to prevent the cell from leaving the cell-trapping device. The cell-trapping area preferably comprises more than 100, further preferred more than 200 and in particular at least 256 cell-trapping microchannels. The cell-trapping microchannels in the cell-trapping area in the microchannel portion in particular have substantially the same size and shape, i.e. have substantially the same dimensions.

The cell-trapping microchannels in the cell-trapping area in the microchannel portion are preferably arranged substantially in parallel at regular intervals in a row along a linear axis through the cell-trapping device. In preferred embodiments, the cell-trapping microchannels are arranged substantially in parallel at regular intervals in a row along an axis substantially parallel to an outer surface of the cell-trapping device.

The microchannel portion preferably further comprises an inlet area and an outlet area, wherein the cell-trapping area is arranged between outlet area and inlet area in the microchannel portion of the cell-trapping device.

The inlet area comprises an inlet, i.e. at least one aperture or opening such as a region constructed for receiving the plurality of cells in a fluid, in particular in form of a suspension of the cells in the fluid such as a culture medium. The amount of cells can be, for example, 1000 cells per μL.

The outlet area is constructed for releasing the fluid such as the culture medium along with untrapped cells which are smaller than the cell-trapping microchannels. The term “untrapped” cells refers to cells which have not been trapped in the cell-trapping microchannels of the cell-trapping device for example because they are too small for being trapped or because the cell-trapping microchannels are already occupied with a cell. The outlet area comprises an outlet, i.e. at least one aperture or opening such as a region, and outlet microchannels for directing the fluid with untrapped cells which are smaller than the cell-trapping microchannels to the outlet of the cell-trapping device.

Preferably, the cell-trapping microchannels proceed into the outlet microchannels, i.e. the cell-trapping device comprises a network of connected microchannels forming flow paths. More specifically, the fluid with untrapped cells which are smaller than the cell-trapping microchannels is preferably directed through the cell-trapping microchannels into the outlet microchannels and to the outlet of the cell-trapping device. Otherwise untrapped cells can be removed by flushing the inlet area with fluid such as with a culture medium.

In preferred embodiments, cell-trapping microchannels proceed into outlet microchannels forming a binary tree-like symmetrical structure. The outlet microchannels proceeding from the cell-trapping microchannels are preferable merged to at most 10 and preferably one single outlet microchannel leading to the outlet of the cell-trapping device.

Preferably, the microchannel portion is formed by two layers, in particular a first layer arranged on a second layer, both forming the cell-trapping area, the inlet area and the outlet area together along their horizontal dimensions.

The term “layer” as used herein refers to a planar material and plate-like material, respectively, with a length and width larger than its height and, hence, with, certain height and perpendicular thereto horizontal dimensions, namely length and width. In particular, a “layered material” has substantially the same height at different points along its horizontal dimensions, i.e. along its length and width direction.

The first layer and the second layer preferably comprise and more preferably consist of polymeric compounds, in particular polymeric organosilicon compounds. Preferably, both first layer and second layer preferably comprise and more preferably essentially consist of polydimethylsiloxane (PDMS). Polymeric compounds are compounds comprising a number of repeated subunits.

The second layer is preferably arranged to trap the cells in the cell-trapping area. The height of the second layer is preferably smaller than the height of the first layer. Preferably, the second layer has a height of up to about 10 μm, more preferably up to about 5 μm and in particular of about 3 μm to about 5 μm. The first layer preferably has a height of up to about 20 μm, more preferably up to about 15 μm and in particular of about 10 μm to about 15 μm.

The cell-trapping microchannels and the outlet microchannels are preferably formed by recesses in the first layer and/or in the second layer. The microchannels are preferably formed by recesses proceeding substantially perpendicular to the horizontal dimensions of first layer and/or second layer, which is substantially parallel to the height direction of the layers, and the microchannels proceed parallel to both horizontal dimensions of first layer and/or second layer. The invention is not limited to microchannels having a substantially rectangular form perpendicular to the main fluid flow direction, i.e. defined by height and width of the microchannels. The microchannels may also have a circular or semicircular form and in these embodiments, width and high means the highest value for width and high of the microchannel, respectively.

The height of a microchannel is preferably understood as the dimension substantially perpendicular to the direction of the horizontal dimensions of the first layer and second layer, i.e. substantially perpendicular to length and width of the layers. The width of a microchannel is in particular a dimension substantially perpendicular to the height and substantially perpendicular to the main fluid flow direction through the channels. A third dimension is the length of a microchannel, i.e. the dimension of the microchannel in the direction of the main fluid flow through the channels which is in particular substantially parallel to the horizontal dimensions of the layers.

The outlet microchannels are preferably formed by recesses in the second layer and in the first layer substantially perpendicular to the horizontal dimensions of the first layer and the second layer which proceed substantially parallel to the horizontal dimensions of first layer and second layer.

The width of the recesses in the outlet microchannels preferably exceeds the width of the cell-trapping microchannels. In preferred embodiments, the recesses in the outlet microchannels have a height of up to about 0.8 to 1×the average cell diameter such as of between about 0.8 to about 1×the average cell diameter. In particular, the height of the recesses in the outlet microchannels is up to about 25 μm. The width is preferably at least 0.8×the average cell diameter and in particular at least 1×the average cell diameter such as at least about 20 μm, preferably at least about 25 μm.

The cell-trapping microchannels preferably have a cell receiving part and a fluid transfer part. The cell receiving part of the cell-trapping microchannels is formed by a plurality of recesses in the first layer and in the second layer suitable to receive one cell each. The recesses of the cell receiving part preferably have a height and a width slightly smaller or at most up to the average cell diameter, in particular 0.8 to 1×the average cell diameter.

The fluid transfer part which allows fluid and optionally untrapped cells smaller than the cell-trapping microchannels to leave the cell-trapping microchannels and proceed to the outlet microchannels is preferably formed by recesses in the second layer only. The width of the recesses in the fluid transfer part is preferably slightly smaller than or up to the average cell diameter, in particular up to about 0.8 to 1×the average cell diameter such as between about 0.8 and about 1×the average cell diameter. The height of the recesses in the fluid transfer part is preferably up to about 0.5×the average cell diameter, more preferably up to about 0.25×the average cell diameter and further preferred about 0.25×the average cell diameter. The height of the recesses in the fluid transfer part is more preferably up to about 10 μm, still further preferred up to about 5 μm and in particular about 3 μm to about 5 μm.

The cell-trapping device preferably further comprises a base portion and/or a cover portion, in particular a base layer and/or cover layer and most preferably base layer and cover layer. The microchannel portion is preferably arranged on the base or cover portion. In particular one of first layer or second layer is arranged on base or cover portion with its horizontal dimensions, i.e. with a surface perpendicular to the height direction of the layer, and the other of first or second layer is then arranged on said layer with its horizontal dimensions.

In embodiments in which the cell-trapping device comprises both base portion and cover portion, the microchannel portion is preferably arranged in between base portion and cover portion.

The base portion and/or the cover portion preferably comprise and in particular essentially consist of glass, in particular base portion and cover portion are a glass layer each.

The cells comprise prokaryotic and/or eukaryotic cells, preferably the cells comprise and in particular consist of human cells. The term “plurality of cells” used herein means more than 10 cells, in particular more than 100 cells and most preferably at least about 256 cells such as about 256 cells. The cells preferably have a diameter of less than 25 μm, more preferably of at most 20 μm like about 15 μm to about 20 μm.

Unless otherwise specified, the term “diameter” as used for cells in the present invention preferably refers to the Feret (or Feret's) diameter at the thickest point of such cell. The Feret diameter is a measure of an object size along a specified direction and can be defined as the distance between the two parallel planes restricting the cell perpendicular to that direction. The Feret diameter can be determined, for example, with microscopic methods. I.e. if the Feret diameters measured for the different directions of the cell differ, the “diameter” referred to in the present patent application always refers to the highest value measured. “Average diameter” refers to the average of “diameter” preferably measured with at least 10 cells, more preferably with at least 30 cells and in particular measured with at least 100 cells.

The term “injectant” as used herein refers to an extracellular material or mixture of extracellular materials, preferably selected from at least one of DNA like plasmid DNA, RNA like synthetic modified RNA, polypeptides or proteins like antibodies, medicinal compounds or bacteria. Polypeptides comprise at least 2 and in particular at least 10 amino acids, wherein proteins usually comprise more than one polypeptide.

Preferably the cell-trapping device is vacuum-based.

At least the microchannel portion, further preferably at least the microchannel portion and the optional cover portion, are transparent for visible light which further allows a visible inspection of the trapped cells. In further preferred embodiments, the cell-trapping device comprising each of microchannel portion, cover portion and base portion is transparent for visible light.

“Visible light” is generally referenced as portion of the electromagnetic spectrum that is visible to the human eye, namely electromagnetic radiation having wavelengths from about 380 to 800 nm, in particular from about 400 to 700 nm.

The term “transparent” as used herein means that the portion or cell-trapping device is capable of transmitting visible light without appreciable scattering or absorption. More specifically, the total transmittance is preferably at least 60%, more preferably more than 65% and especially preferably more than 80% at the thickness of the respective material as suitable for the portion or for the cell-trapping device of the present invention. Such transmittance values are preferably achieved at a thickness of up to 10 μm, preferably up to 100 μm, more preferably up to 1000 μm or even up to 5000 μm. The transmittance and transmission, respectively, can be determined by conventional methods known to the skilled person, in particular in accordance with ASTM D 1003 by conventional spectrophotometer or hazemeter.

The haze value of the portion(s) or cell-trapping device is, in particular, less than 40%, preferably less than 30%, more preferably less than 10% at a suitable thickness of the portion(s) or cell-trapping device. Such haze value is usually measured at a thickness of up to 10 μm, preferably up to 100 μm, more preferably up to 1000 μm or even up to 5000 μm. The haze value is a measure of the haze of transparent materials. This value describes the proportion of the transmitted light that is scattered or reflected by the irradiated material. The internal transmittance, i.e. considering possible absorption, is in particular above 65%, preferably above 70%, further preferred of above 85% at a thickness of up to 10 μm, preferably up to 100 μm, more preferably up to 1000 μm or even up to 5000 μm.

In a second aspect, the present invention provides a method of preparing a cell-trapping device as described above, preferably including soft lithography. The method of the present invention comprises steps of:

(i) providing a forming member with protuberances that correspond in size and shape to the dimensions of the microchannels in the microchannel portion, in particular of the cell-trapping microchannels and the outlet microchannels;

(ii) applying a mixture comprising a polymeric compound, in particular a polymeric organosilicon compound and more preferably a mixture comprising PDMS, onto the forming member and curing the mixture for forming the microchannel portion;

(iii) optionally applying the microchannel portion to at least one of base portion or a cover portion along the horizontal dimensions of the microchannel portion.

Step (i) preferably comprises steps of:

(a) transferring an ultraviolet light (UV) mask adapted to obtain the second layer to a spin-coated negative photoresist film with a depth corresponding to the height of the second layer, followed by irradiation with UV light, heating, and removing the unexposed area of photoresist for obtaining a preliminary forming member with protuberances; and

(b) repeating step (a) with a spin-coated negative photoresist film applied to the preliminary forming member with a depth on the protuberances of the preliminary forming member corresponding to the height of the first layer and a UV mask adapted for the first layer.

The ultraviolet (UV) light masks can be prepared by printing on a transparent substrate with a high resolution printer. The spin-coated negative photoresist film is preferably prepared by spin coating epoxy-based negative photoresist, in particular SU-8 negative photoresist, on a silicon wafer and subsequent heating preferably on a hotplate by increasing the temperature from room temperature, i.e. about 25±2° C. to about 95° C. in about 5 min and maintaining a temperature of about 95° C. for about 3 min.

Heating following UV irradiation in step a) and b) is preferably carried out on a hotplate by heating from room temperature of about 25±2° C. to about 80° C. in about 5 min and maintaining a temperature of about 80° C. for about 2 min. Preferably, the unexposed photoresist is removed using the SU-8 developer. Ultraviolet light as used in particular refers to electromagnetic radiation having a wavelength from about 315 to about 380 nm, in particular of about 365 nm.

In step (ii) the mixture preferably comprises PDMS and a curing agent such as in a weight ratio of about 10:1. The mixture is preferably degassed before curing in particular by applying a vacuum. Curing is preferably carried out in an oven.

The method for preparing the cell-trapping device preferably further comprises steps after step (ii) and before step (iii) of peeling the microchannel portion off from the forming member, providing an outlet and adjusting the size of the microchannel portion, preferably under the microscope. The outlet is preferably provided by means of a sharpened syringe needle.

Step (iii) preferably includes applying the microchannel portion to a base layer and/or a cover layer which are glass layers. Step (iii) is preferably carried out by means of plasma bonding, in particular in an oxygen plasma.

In a third aspect, an apparatus for high-throughput microinjection of an injectant into a plurality of cells with an average diameter of at most 25 μm is provided with the present invention. The apparatus comprises:

a cell-trapping device as described above;

an injection needle with a tip arranged to be stuck into the cells trapped in the cell-trapping area of the cell-trapping device to inject the injectant into the trapped cells.

The high-throughput microinjection is preferably an automated high-throughput microinjection.

The cells preferably comprise and in particular consist of human cells. Preferably, “plurality of cells” means more than 100 cells, and further preferably more than about 200 cells and in particular more than about 256 cells such as about 256 cells. The cells preferably have an average diameter of less than about 25 μm, more preferably at most about 20 μm like about 15 μm to about 20 μm. The term “stuck into the cells” includes stuck into the cell as such or into specific cell compartments. In particular, “stuck into the cell” includes stuck into the cytoplasm or into the cell nucleus.

The injection needle is a capillary needle and preferably in the form of a micropipette. The injection needle preferably has an average tip diameter of up to about 0.7 μm, further preferably of up to about 0.5 μm such as of about 0.5 μm. The injection needle preferably comprises and in particular consists of glass. The outer injection needle diameter can be about 0.5 mm to about 2 mm, preferably about 1 mm. The inner injection needle diameter can be about 0.1 mm to about 1.5 mm, preferably about 0.5 mm.

The injection needle is preferably either provided with a bent form or is bent to a bent form before being stuck into the cells. In preferred embodiments, the injection needle is bent to a bent form before being stuck into the cells. The term “bent form” of an injection needle as used herein means that the injection needle has a needle tilt angle or is bent to a needle tilt angle α compared to the straight form of the injection needle of more than 10°, preferably of more than 30° and in particular of more than 70° such as between more than 70° and 90°.

The injection needle preferably has an interior space filed with the injectant. The injectant is preferably an extracellular material or mixture of extracellular materials, preferably DNA like plasmid DNA, RNA like synthetic modified RNA, polypeptides or proteins. The cell-trapping device is preferably transparent for visible light. Further preferred, the cell-trapping device is vacuum-based.

The apparatus of the present invention preferably further comprises at least one of and preferably each of:

a device carrier member;

a needle holding member;

a control unit for guiding the injection needle to the trapped cells;

a cell-detection unit to detect the cells and to generate a signal for initiating the injection;

an pressure-based microinjector;

anti-vibration means.

The term “device carrier member” refers to means for carrying the cell-trapping device. The device carrier member in particular has a device carrying surface directed to the cell-trapping device and suitable to carry the cell-trapping device directly or indirectly, i.e. with or without direct contact between the device carrying surface and the cell-trapping device. Indirect contact, thus, means that there is no direct contact between cell-trapping device and device carrying surface, in particular that a dish unit preferably comprising a petri dish into which the cell-trapping device is placed is located between cell-trapping device and device carrying surface of the device carrier member.

Preferably, the cell-trapping device is carried by the device carrier member such that the base portion, in particular the base layer is directed towards the device carrying surface of the device carrier member and the cover portion is on the opposite side of the cell-trapping device.

The device carrier member can have any suitable form and dimensions as long as it is suitable to carry and further preferably to carry and to move the cell-trapping device. The device carrier member preferably has means for receiving and/or fixing elements on the device carrying surface like the cell-trapping device or a petri dish into which the cell-trapping device is placed. The means for receiving or fixing preferably comprise at least 2 clamps. In one embodiment, a petri dish into which the cell-trapping device is placed is fixed on the device carrying surface of the device carrier member.

The device carrier member is preferably arranged such that it can move the cell-trapping device. The device carrier member can either move the cell-trapping device by movement of the device carrier member or by movement of its device carrying surface. Preferably, the device carrier member can move the cell-trapping device at least along a plane parallel to level ground, preferably in directions of two coordinate axes in a two-dimensional orthogonal coordinate system in the plane parallel to level ground referenced as “X-Y plane”. In particular, the device carrier member is arranged such that it can move the cell-trapping device during the microinjection along the X direction and along the Y direction in the X-Y plane. Such device carrier member is also referenced herein as “X-Y device carrier member”.

Movement or motion in the X direction means motion in an axis preferably parallel to level ground. Motion in the Y direction means motion in an axis perpendicular to the direction of the X axis and preferably also parallel to level ground.

Generally, the device carrying surface of the device carrier member, in particular of the X-Y device carrier member, is in a horizontal position that is substantially parallel to the X-Y plane.

The needle holding member refers to means suitable for holding the injection needle, in particular means onto which the injection needle is mounted in particular detachably with the end opposite to the tip.

The needle holding member can have any suitable form and dimensions as long as it can hold, in particular hold the end of the needle opposite to the tip and further preferably hold and move it. Preferably, at least a portion of the needle holding member with the end of the injection needle opposite to the tip and/or the needle holding member are movable in particular perpendicular to the X movement and Y movement of the device carrier member, i.e. along a Z direction, which is referenced herein as “Z needle holding member”, and in particular perpendicular to the X-Y plane. In an embodiment, X direction and Y direction of the cell-trapping device and Z direction of the needle holding member represent the axes of a Cartesian coordinate system. Z direction preferably means a movement or motion along an axis substantially perpendicular to a plane parallel to level ground that is up and down.

The end of the capillary needle opposite to the tip is preferably hold by and in particular mounted on a surface of the needle holding member arranged such that the plane parallel to said surface is substantially perpendicular to the plane parallel to the device carrying surface of the device carrier member, in particular such that the plane parallel to said surface is substantially perpendicular to the X-Y plane.

The apparatus preferably further comprises a control unit for guiding the tip of the injection needle to the trapped cells in particular based on the information provided by a cell-detection unit. The control unit usually comprises a computer. The control unit preferably further comprises means referenced herein as motion controller for controlling the position of the cell-trapping device and/or of at least a portion of the needle holding member, in particular for moving the cell-trapping device in X direction and in Y direction in the X-Y plane and/or for moving at least a portion of the needle holding member in Z direction perpendicular to the X-Y plane. In particular, the control unit with the motion controller allows for moving the cell-trapping device in said X direction and in said Y direction and for moving at least a portion of the needle holding member in said Z direction.

The apparatus preferably further comprises a cell-detection unit to detect the trapped cells and to generate a signal for initiating the microinjection. The cell detection unit in particular comprises a vision detector and microscopic means. The vision detector is preferably a CCD camera and the microscopic means is preferably a microscope.

The cell-detection unit preferably further comprises a light source providing illumination to the microscopic means. In embodiments of the apparatus of the present invention, the vision detector such as the CCD camera is mounted on the microscopic means such as a microscope which is connected to the light source.

The cell-detection unit is preferably arranged on top of the cell-trapping device. The expression “on top” of the cell-trapping device means that the cell-detection unit faces the surface of the cell-trapping device which is opposite to the surface of the cell-trapping device facing towards the device carrying surface of the device carrier member. The cell-detection unit, in particular the microscopic means and/or the vision detector, is preferably arranged such that it is movable perpendicular to the X-Y plane, i.e. in Z direction.

The apparatus in particular further comprises a pressure-based microinjector, which is an injector providing pressure in particular negative pressure to the cell-trapping device for trapping the cells and/or positive pressure to the injection needle. The microinjector is in particular connected to the injection needle and/or the cell-trapping device, in particular the outlet of the cell-trapping device. The microinjector is in particular embodiments connected to the injection needle and the outlet of the cell-trapping device and provides negative pressure to the outlet of the cell-trapping device for trapping the cells and positive pressure to the injection needle.

The apparatus preferably further comprises means for reducing vibration referenced herein as “anti-vibration means”, in particular an anti-vibration member such as a table or plate onto which the device carrier member with the surface opposite to the device carrying surface and/or the needle supporting member can be placed.

Ina fourth aspect, the present invention provides a method for microinjection of an injectant into a plurality of cells having an average diameter of at most 25 μm comprising steps of:

(i) providing an apparatus as described above;

(ii) introducing a plurality of cells into the cell-trapping device;

(iii) trapping the cells in the cell-trapping microchannels in the cell-trapping area of the cell-trapping device such that a cell-trapping microchannel traps one cell;

(iv) inserting the injection needle with the tip into the cell-trapping area of the cell-trapping device and injecting the injectant subsequently into a plurality of trapped cells.

Preferably, step (iii) is carried out such that least 60%, further preferred at least 70% and in particular at least 80% of the cell-trapping channels in the microchannel portion of the cell-trapping device tap one cell.

The microchannel portion of the cell-trapping device preferably comprises an inlet area having an inlet constructed for receiving the plurality of cells in a fluid and an outlet area having outlet microchannels for directing the fluid along with untrapped cells smaller than the cell-trapping microchannels to an outlet for releasing the fluid along with the untrapped cells, wherein the cell-trapping area is arranged between outlet area and inlet area; and wherein the cell-trapping device comprises at least 200 cell-trapping microchannels in the cell-trapping area.

Step (ii) preferably comprises applying the cells in a fluid to the inlet of the cell-trapping device, preferably by means of a liquid transfer pipette. The cells are preferably suspended in a fluid in particular in a culture medium. Preferably, the amount of cells is about 1000 cells per μL. Preferably, the method further comprises a step after step (i) and before step (ii) of loading fluid such as culture medium through the outlet into the cell-trapping device in particular by connecting to outlet to a syringe with fluid such as culture medium.

Step (iii) is preferably carried out by applying a negative pressure at the outlet of the cell-trapping device for cell trapping, preferably a negative pressure of about 100 Pa to less than about 400 Pa such as of about 124.6 Pa to less than about 400 Pa, further preferably of less than about 249 Pa. Preferably, a negative pressure of between about 5% and about 25% of the negative pressure applied for trapping the cells is maintained after cell trapping for holding the trapped cells in the cell-receiving part, in particular a negative pressure of about 24.9 Pa. The cell trapping time is preferably about 10 min for obtaining a trapping efficiency of at least 70%, preferably of at least 75% and in particular of more than 80%. The trapping efficiency is the ratio of the number of cell-trapping microchannels with a trapped cell to the total number of cell-trapping microchannels.

The injection needle preferably has a tip diameter of up to about 0.7 μm, preferably of up to about 0.5 μm such as of about 0.5 μm. Preferably, inserting the injection needle into the cell-trapping area in step (iv) includes bending the injection needle while inserting the tip into the cell-trapping area of the cell-trapping device for obtaining a needle tilt angle α of more than 10°, preferably of more than 30° and in particular of more than 70°.

Inserting the injection needle with the tip is preferably carried out by means of moving the cell-trapping device in the direction of the tip such that the tip reaches the cell-trapping area in front of a trapped cell, preferably by movement by means of the device carrier member such as by movement of the device carrier member or of the device carrying surface of the device carrier member, in particular a X-Y device carrier member, in the direction of the tip in particular controlled by the control unit. The use of a straight-line path, namely an X-Y path along an X and perpendicular thereto an Y direction, can minimize the damage to the cells during injection. Preferably, the cell-trapping device is moved toward the tip such that the tip penetrates the cell membrane of the trapped cell or reaches a cell compartment in the trapped cell. The injection needle is preferably fixed.

Step (iv) may include a further step of aligning the trapped cell with the tip before injection.

The injection of the injectant into the cell in step (iv) is preferably carried out by applying a positive pressure for a predetermined time period depending on the amount of injectant to be injected.

Step (iv) preferably comprises steps of:

(a) inserting the injection needle with the tip into the cell-trapping area of the cell-trapping device; optionally moving the cell-trapping device in X direction and/or Y direction and/or moving the needle holding member with the end of the needle opposite to the tip in Z direction perpendicular to the X direction and to the Y direction, and in particular perpendicular to the X-Y plane;

(b) aligning a first trapped cell with the tip and moving the cell-trapping device in the direction of the trapped cell in particular by moving the cell-trapping device in a direction to the tip and/or perpendicular to said direction such that the tip is stuck into the first trapped cell;

(c) injecting the injectant into the first trapped cell;

(d) moving the cell-trapping device away from the tip and to a second trapped cell, in particular by moving the cell-trapping device in a direction opposite to the tip in particular back to its original position, and subsequently perpendicular to said direction such that the tip is substantially in front of the second trapped cell,

(d) aligning the second trapped cell with the tip and moving the cell-trapping device in the direction of the second trapped cell, in particular by moving the cell-trapping device in a direction to the tip and/or perpendicular to said direction such that the tip is stuck into the second trapped cell;

(e) injecting the injectant into said second trapped cell; and

(f) repeating steps (d) to (e) with a third and any further trapped cell until all trapped cells have received the injectant.

One of the direction to or opposite to the tip and the direction perpendicular thereto being the X direction and the other being the Y direction.

In preferred embodiments, the method includes a further step after inserting an injection needle with the tip into the cell-trapping area and before injecting the injectant into a plurality of trapped cells such as before step (b) and/or (d) referenced above of identifying whether there is a further target cell in the cell-trapping area.

A target cells is a cell and region, respectively, with a specific correlation of edge information compared to a template image of one predetermined template cell and region, respectively. This step contributing to an advantageously automated microinjection preferably includes an image processing by means of a control unit and a cell-detection unit based on template matching of edge information, in particular it includes:

searching the position of an uninjected trapped target cell by calculating the correlation of edge information between the template image and each pattern region on a sample image;

determining whether the correlation is larger than a set threshold; and

if this condition is met, proceeding with the injection of the injectant into the cell.

The term “template image” refers to an image of a selected region with a cell, respectively, used to locate target cells . “Sample image” refers to an image of a region, respectively, in which untrapped target cells are to be identified.

In further preferred embodiments, the further step of identifying a target cell in the cell-trapping area comprises:

providing a template image by adjusting the position of the microscopic means like the microscope to bring the cell-receiving portion of the cell-trapping area into focus; capturing an image containing both template cell and injection needle such as micropipette and inputting the region of the cell and the position of the tip of the injection needle on the captured image;

providing a sample image of the cell-trapping area and preprocessing the sample image comprising reprocessing with a low-pass Gaussian filter, extracting the edge information using the Sobel edge detector followed by morphological operation;

processing the template image using the same procedure for obtaining the edge information of the template image;

locating the central position of the region on the sample image, where the edge information is similar to that of the template image;

calculating the correlation of edge information between the template image and each pattern region on the sample image;

determining whether the correlation is larger than a threshold;

if this condition is met, the center of the matched pattern region in the sample image is used to define the cell position.

If the condition is not met or if all target cells are injected, the injection process stops.

The volume of injectant in step (iv) is preferably controlled by the injection pressure and injection time.

The method of the present invention preferably allows for an injection efficiency of more than 50%, in particular of at least 80% and most preferably of at least 85%, The injection efficiency is defined as the ratio of the number of the cells comprising the injectant after microinjection to the number of the total injected cells, namely

$$Injection efficiency = \frac{no . of cells comprising injectant after injection}{no . of injected cells}$$

The method of the present invention preferably allows for a survival rate of at least 60%, in particular of at least 70% and in particular of at least 80%. The survival rate is defined as the ratio of the number of cells comprising the injectant 24 h after microinjection to the number of cells comprising the injectant right after microinjection, namely

$$Survival rate = \frac{no . of living cells comprising injectant 24 h after injection}{no . of living cells comprising injectant right after injection}$$

Preferably, at least about 30 cells per min, in particular at least about 35 cells per min such as about 35 cells per min are injected with the method of the present invention. The precision is preferably about 0.2 μm. The cells preferably comprise and in particular consist of human cells with an average diameter of preferably less than about 25 μm, more preferably at most about 20 μm like about 15 μm to about 20 μm. In particular, more than 100 cells, more preferably at least 200 cells, and in particular at least about 256 cells can be injected with the method of the present invention.

The injectant is preferably selected from at least one of DNA like plasmid DNA, RNA like synthetic modified RNA, polypeptides or proteins.

The provided cell-trapping device and apparatus are especially suitable to be employed in the automated and high-throughput microinjection of exogenous macromolecules into small cells with a diameter of less than 25 μm which is the typical size of many human cells and even for cells with a diameter below 15 μm and adherent cells. Most of the known methods can only be used for injection into cells with a diameter above 50 μm, such as mouse embryo injection systems and zebrafish embryo injection systems.

The cell-trapping device, apparatus and method of the present invention in particular allow for a rapid and highly reproducible microinjection into a plurality of individual small cells with high productivity and accuracy. Moreover, the cell survival rate following the microinjection with the cell-trapping device and/or apparatus of the present invention is advantageously high and the target cells can be easily and accurately located due to the specific microchannel structure of the cell-trapping device and, thus, are suitable to overcome the problem of the irregular morphology of human cells which usually tremendously increases the difficulty in recognizing and locating these cells. In particular, the cell-trapping device, apparatus and method of the present invention allow for an exceptionally high-throughput and efficiency of up to about 35 cells/min which is not achievable with many known injection systems in particular when used for injection into smaller and/or adherent cells.

The cell-trapping device of the present invention can individually trap a large amount of cells, i.e. more than about 200 cells within 10 min and in particular at least about 256 cells. In particular due to the cell-trapping device of the present invention along with the use of an injection needle with a needle tilt angle of more than 30°, in particular of more than 70°, the microinjection task can be further simplified as it can be achieved in a single-axis motion. Exogenous materials such as plasmid DNA and synthetic modified RNA can be successfully delivered into cells for inducing desirable phenotypic changes.

### BRIEF DESCRIPTION OF THE DRAWINGS

FIGS. 1A, 1B, and 1C illustrate embodiments of the apparatus of the present invention, wherein, FIG. 1A is a schematic diagram of an apparatus according to one embodiment of the present invention; FIG. 1B is a schematic diagram of an arrangement of the cell-trapping device, device carrier member, needle holding member and anti-vibration means in one embodiment of the apparatus of the present invention with a bent form of the injection needle having a needle tilt angle α of more than 10°; and FIG. 1C illustrates an arrangement of the cell-trapping device, device carrier member, needle holding member and anti-vibration means in one embodiment of the apparatus of the present invention wherein the device carrier member can move the cell-trapping device in X direction and Y direction and at least a portion of the needle holding member can be moved in Z direction.

FIGS. 2A, 2B, 2C, 2D, and 2E illustrate embodiments of the cell-trapping device of the present invention, wherein FIG. 2A is a top view and illustrates one embodiment of the cell-trapping microchannels in the cell-trapping device with a cell receiving part and a fluid transfer part; FIG. 2B is a front view and illustrates one embodiment of the cell-trapping microchannels in the cell-trapping device; FIG. 2C is a top view of the microchannel portion of the cell-trapping device and illustrates an embodiment with inlet area, cell-trapping area and outlet area; FIG. 2D illustrates one embodiment of the cell-trapping device of the present invention with full cell loading; and FIG. 2E is a side view and schematic representation of one embodiment of the cell-trapping device with top portion and cover portion and microchannel portion with first layer and second layer.

FIGS. 3A, 3B, 3C, and 3D illustrate embodiments of the method of the present invention for microinjection of an injectant into a plurality of cells, wherein FIG. 3A illustrates the introduction of cells suspended in a fluid into the cell-trapping device; FIG. 3B illustrates the cell trapping in the cell-trapping area in the cell-trapping device; FIG. 3C illustrates the step of inserting the injection needle into the cell-trapping area of the cell-trapping device; and FIG. 3D illustrates an automated cell injection.

FIG. 4 shows a flowchart of embodiments of the method of the present invention for microinjection of an injectant into a plurality of cells including a cell recognition strategy.

FIG. 5 illustrates the coordinate frames of vision detector and device carrier member in embodiments of the apparatus of the present invention.

FIG. 6 shows a visual-guided position control scheme for automated cell injection of one embodiment of the present invention.

FIGS. 7A, 7B, 7C, and 7D illustrate the identification of target cells in an embodiment of the method of the present invention for microinjection of an injectant into a plurality of cells, wherein FIG. 7A shows a template image; FIG. 7B shows an original (sample) image; FIG. 7C shows the reprocessed image; and FIG. 7D shows the recognition result after applying an edge template matching algorithm.

FIG. 8 illustrates an injection path plan that includes three paths for aligning a cell with the micropipette, moving the cell toward the micropipette, and moving the cell back to its original position.

FIG. 9 illustrates the method for preparing the cell-trapping device in one embodiment of the present invention.

FIGS. 10A, 10B, 10C, and 10D show simulation results of the fluid flow velocity for single cell trapping, wherein FIG. 10A shows the simulated region in case of empty cell-trapping microchannels (top view); FIG. 10B shows the simulated region in case of empty cell-trapping microchannels (side view); FIG. 10C shows the simulated region in case of occupied cell-trapping microchannels (top view); and FIG. 10D shows the simulated region in case of occupied cell-trapping microchannels (side view).

FIGS. 11A, 11B, 11C, and 11D illustrate a microinjection into HFF cells with the method of the present invention, wherein FIG. 11A shows the trapped HFF cell moving into the direction of the micropipette; FIG. 11B illustrates the tip of the micropipette penetrating the cell membrane of the trapped HFF cell; FIG. 11C shows a further step of moving the cell-trapping device in a direction opposite to the tip and subsequently perpendicular to said direction such that the tip reaches the next trapped cell; and FIG. 11D shows the trapped HFF cell moving into the direction of the micropipette.

FIG. 12 shows the HFF cell trapping by a cell-trapping device of the present invention.

FIGS. 13A, 13B, 13C, and 13D show photographs of HFF cells before and after the automated injection of TRITC-Dextran, wherein FIG. 13A is a bright field image; FIG. 13B shows a fluorescent image of the trapped HFF cells before microinjection; FIG. 13C is a bright field image; and FIG. 13D shows a fluorescent image of the trapped HFF cells after microinjection of TRITC-Dextran.

FIGS. 14A, 14B, 14C, and 14D show images of the HFF cells after injection of TRITC-Dextran and incubation for 24 h, wherein FIG. 14A and FIG. 14B are a bright field images; and FIG. 14C and FIG. 14D are fluorescent overlaid images. The arrows indicate dead cells.

FIG. 15 illustrates the effect of the negative pressure on the cell-trapping efficiency.

### DESCRIPTION OF THE INVENTION

For the purposes of promoting an understanding of the principles of the invention, reference will now be made to the embodiments illustrated in the drawings and specific language will be used to describe the same. It will nevertheless be understood that no limitation of the scope of the invention is thereby intended. A person skilled in the art will understand that features specifically mentioned for the cell-trapping device in the context of preferred embodiments are also applicable in the apparatus of the present invention and vice versa.

The usage of words indicating preferences, such as “preferably,” refers to features and aspects that are present in at least one embodiment, but which are optional for some embodiments.

The technical terms used in the present patent application have the meaning as commonly understood by a respective skilled person unless specifically defined otherwise.

As used herein and in the claims, “comprising” means including the following elements but not excluding others. “Essentially consisting of” means that it consists of the respective element(s) along with usually and unavoidable impurities. “Consisting of” means that something solely consists of, i.e. is formed by respective element(s).

Any reference to prior art contained herein is not to be taken as an admission that the information is common general knowledge, unless otherwise indicated.

And although various specific quantities such as specific values of parameters may be stated herein, such specific quantities are presented as examples only, and further, unless otherwise noted, are approximate values, and should be considered as if the word “about” prefaced each quantity.

The invention refers in an aspect to an apparatus for high-throughput microinjection of an injectant into a plurality of cells with an average diameter of at most 25 μm comprising:

a cell-trapping device; and

an injection needle with a tip arranged to be stuck into the cells trapped in the cell-trapping area of the cell-trapping device to inject the injectant into the trapped cells. The cell-trapping device comprises a microchannel portion having formed therein a cell-trapping area comprising a plurality of cell-trapping microchannels configured to trap one cell per cell-trapping microchannel.

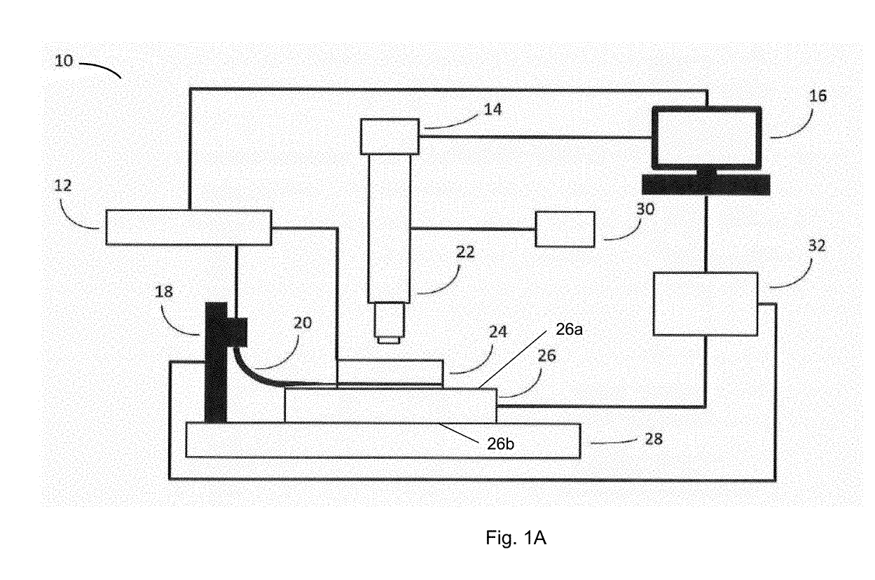

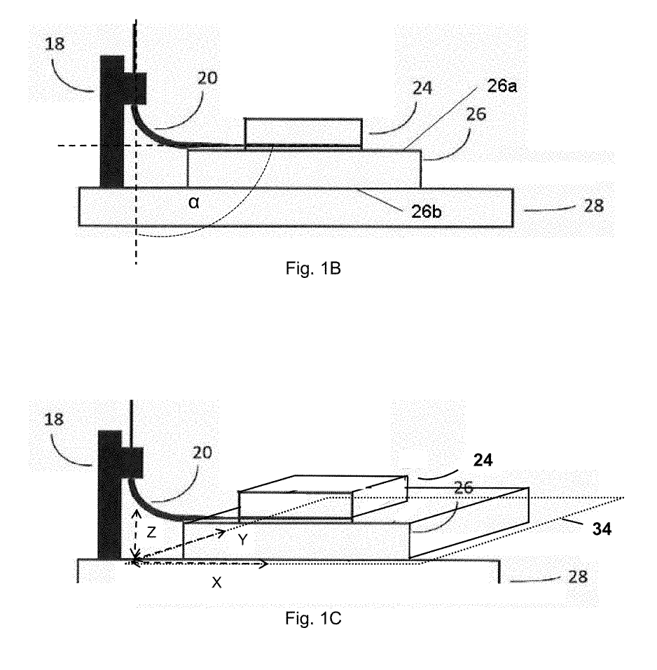

According to FIG. 1A the apparatus of the present invention 10 comprises in an embodiment a cell-trapping device 24. Automated microinjection is conducted by a device carrier member 26 namely a motorized X-Y device carrier member and a needle holding member 18 namely a motorized needle holding member. A cell-trapping device 24, namely vacuum-based cell-trapping device, is placed on the device carrier member 26. A pressure-based microinjector 12 is connected to the outlet of the cell-trapping device 24, providing sufficient and adjustable negative pressure for cell trapping.

The microinjector 12 also provides adjustable positive pressure to an injection needle 20, namely a bent micropipette, which is mounted on a needle holding member 18, which is a Z needle holding member. The diameter of the tip of the injection needle 20 is 0.5 μm. The automated cell injection system also includes a cell-detection unit: a vision detector 14, which is a CCD camera, is mounted on microscopic means 22, namely a microscope, which is placed on top of the cell-trapping device 24. A light source 30 provides illumination to the microscopic means 22. A control unit 16, which comprises a personal computer, can control the position of the X-Y device carrier member and Z needle holding member through the motion controller 32. The device carrier member 26 has a device carrying surface 26a facing towards the cell-trapping device and a surface 26b opposite to said surface. The control unit 16 is also connected to the microinjector 12 and can trigger the positive pressure applied to the bent micropipette for cell injection. In addition, the CCD camera as vision detector 14 combined with an image processing method can be coupled to the control unit 32 to locate the target cells, automating the manipulation of the entire apparatus 10. The automated cell injection apparatus 10 is installed on anti-vibration means 28 in form of an anti-vibration table.

As shown in FIG. 1B, the micropipette is bent to a bent form with a needle tilt angle α compared to the straight form of the micropipette of at least 10°.

FIG. 1C refers to an embodiment of the apparatus of the present invention comprising the cell-trapping device 24 carried by the device carrier member 26 which is a X-Y device carrier member with a device carrying surface 26a of the device carrier member, wherein surface 26b faces towards the anti-vibration means 28. The apparatus comprises a needle holding member 18 in form of a Z needle holding member holding an injection needle 20 in form of a micropipette. The X-Y plane 34 is the plane parallel to level ground. The X-Y device carrier member can move the cell-trapping device 24 in the X and Y direction. The needle holding member or a portion thereof can be moved in Z direction.

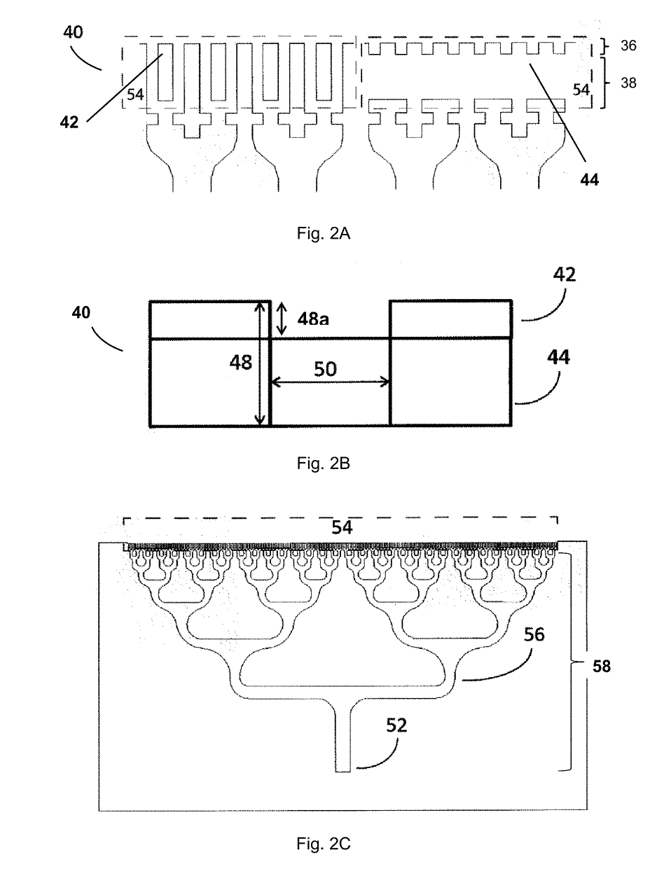

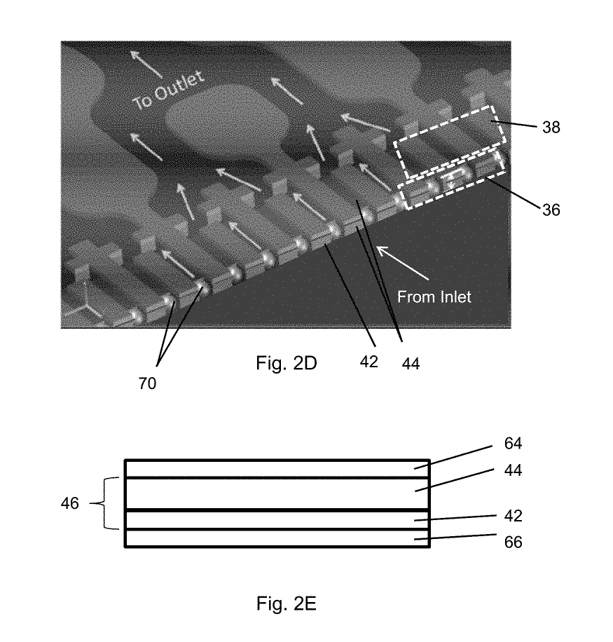

FIG. 2A to FIG. 2C show an embodiment of the cell-trapping device of the present invention with a microchannel network with cell-trapping microchannels 40. The microchannel portion is formed by a first layer 44 and a second layer 42, namely a thin second layer 42 (3-5 μm) and a thick first layer 44 (10-15 μm) . The microchannel network includes a cell-trapping area 54, which consists of 256 cell-trapping microchannels 40. The two layers display the same binary tree-like branching structure, except at the cell-trapping area 54. The dimensions of the recesses forming the cell-trapping microchannels 40 (including both second and first layer) , namely height 48 and width 50, in the cell receiving part 38 are the same or slightly smaller than the size of the cells 70 to be trapped. In this embodiment, width 50 and height 48 of the recesses forming the cell receiving part are 0.8-1×the average cell diameter. In this way, a relatively large friction force can be produced to hold the cell tightly during injection. A small channel may impose large stress on the cell, which degrades cell vitality. The thin second layer 42 is designed to prevent cells from entering the fluid transfer part 38. The negative pressure provided by the microinjector 12 generates a current that flows from the cell-trapping area 54 to the outlet 52 in the outlet area 58 with outlet microchannels 56. The cells move toward the cell-trapping area 54 along with the fluid but cannot pass through the fluid transfer part of the cell-trapping microchannels 46 because of size exclusion. An adherent cell is so flexible that it can easily squeeze through the fluid transfer part if the height is not small enough. Thus, the width 50 of the recesses forming the microchannels in the fluid transfer part is smaller or equal to the average diameter of the cells 70 to be trapped, wherein the height 48a is up to about 0.25×the average diameter of the cells 70 to be trapped.

In the embodiment shown in FIG. 2E the cell-trapping device includes a microchannel portion 46 with a second layer 42 and a first layer 44, further it includes a base portion 66 and a cover portion 64.

In an embodiment of the cell-trapping device of the present invention, the cell-trapping device is transparent for the visible light for clear observation. In one embodiment, the material used for the first and the second layer is poly(dimethylsiloxane) (PDMS) and the fabrication method used is soft lithography. The forming member with a microfluidic channel network is created by transferring the shadow ultraviolet (UV)-mask to the spin-coated negative photoresist film that displays a certain depth. PDMS mixed with the included curing agent at a 10:1 ratio is degassed and poured onto the forming member. An optically transparent replica is prepared to obtain the reverse structure of the forming member after curing. Holes are then punched to provide the outlet area by using a sharpened syringe needle, and the microchannel portion is trimmed to the proper size under the microscope. The bottom glass layer, typically a cover slip, is bonded to the microchannel portion in the oxygen plasma to form an irreversible seal.

In a further aspect, the present invention provides a method for microinjection of an injectant into a plurality of cells having an average diameter of at most 25 μm comprising steps of:

(i) providing an apparatus as claimed in claim 10;

(ii) introducing a plurality of cells into the cell-trapping device;

(iii) trapping the cells in the cell-trapping microchannels in the cell-trapping area in the microchannel portion of the cell-trapping device such that a cell-trapping microchannel traps one cell;

(iv) inserting an injection needle with the tip into the cell-trapping area in the microchannel portion of the cell-trapping device and injecting the injectant subsequently into a plurality of trapped cells.

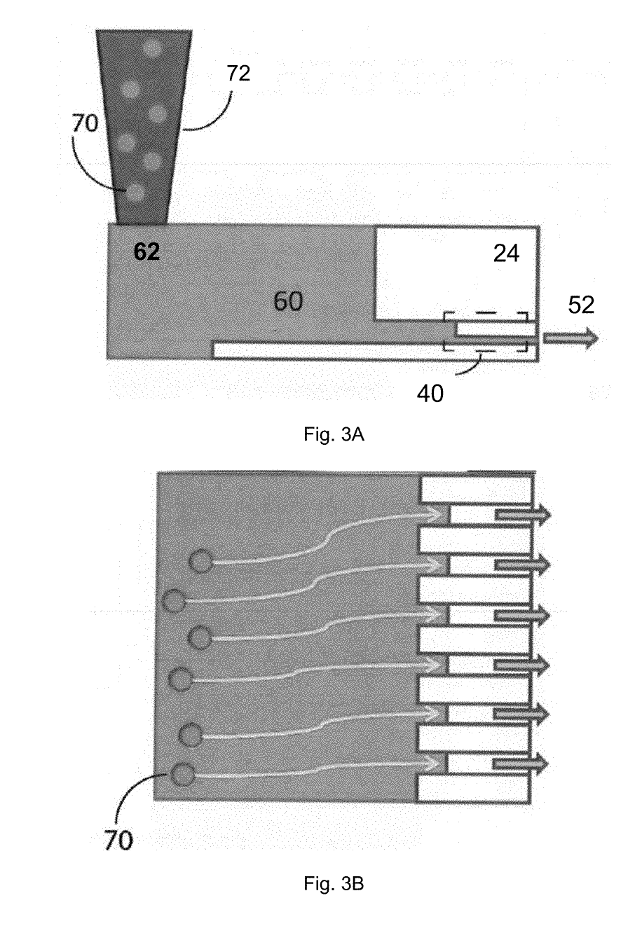

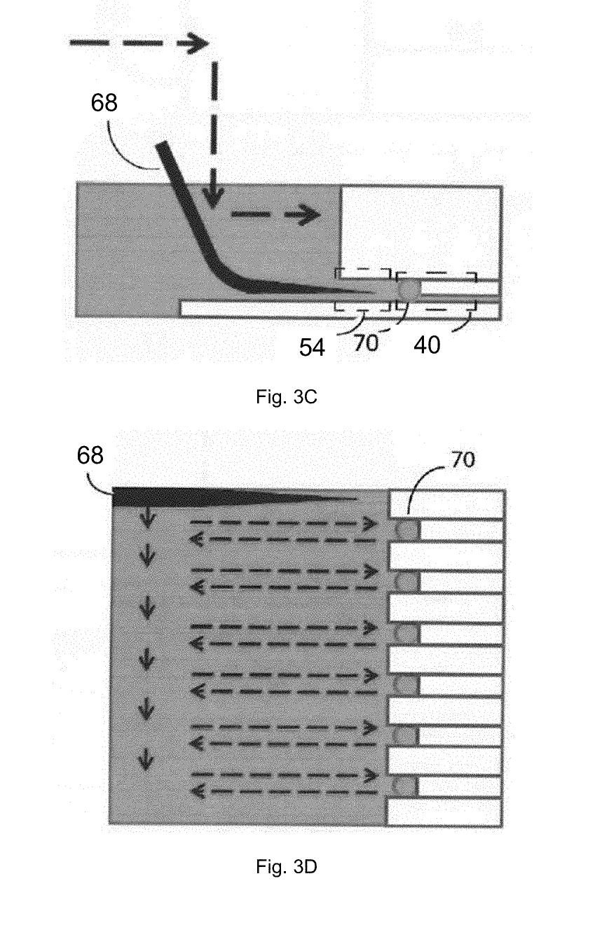

An embodiment of the method of the present invention for microinjection is shown in FIG. 3A to 3D. Cells 70 suspended in a fluid are introduced near the inlet 62 in the inlet area 60 of the cell-trapping device 24 by using a liquid transfer pipette 72. A negative pressure is applied at the outlet 52 of the cell-trapping device 24, creating a fluid flow toward the cell-trapping microchannels 40 (FIG. 3A). The cells 70 are then transported toward the cell-trapping microchannels 40 (FIG. 3B). After the cell-trapping device is fully loaded, the injection needle 20 in form of a micropipette is bent and inserted with its tip into the cell-trapping area 54 (FIG. 3C). The automated cell injection procedure then starts (FIG. 3D). First, a template of cell image is inputted into the system by selecting the region of interest. The edge information of the selected region is used as a template to locate other target cells. The system will search through the whole image. If the correlation of the sample image and the template image is larger than the set threshold, the sample image will be considered as a target cell.

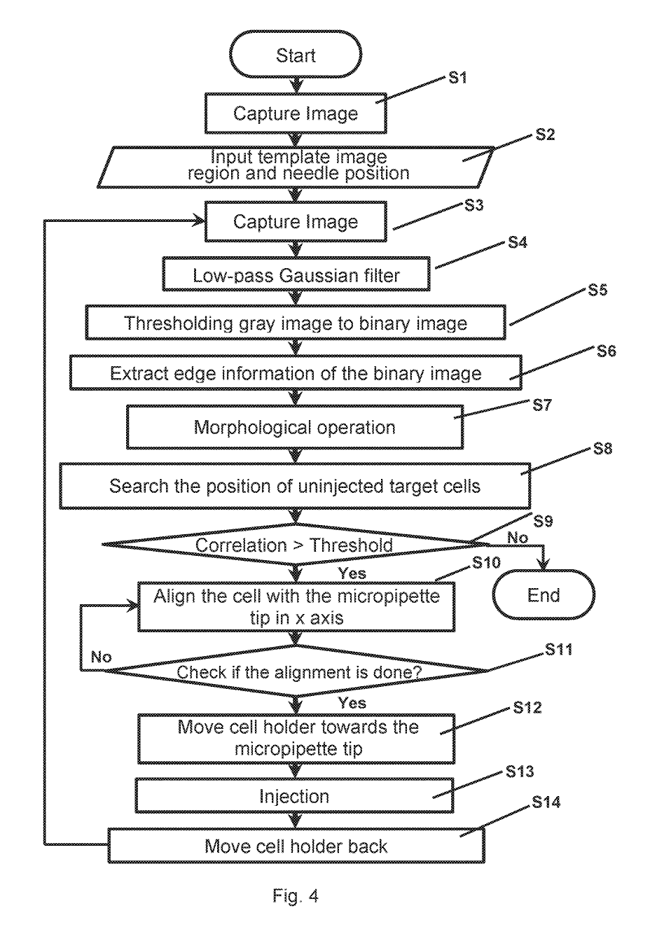

FIG. 4 illustrates one embodiment of steps of the method of the present invention in which the microinjection is automated. After the cells are trapped in the cell-trapping microchannels and after loading the micropipette into the cell-trapping area, the position of the microscope is adjusted to bring the inlet of the cell-trapping area into focus. An image containing both trapped cells and micropipette is captured (S1). The operator inputs the region of a target cell on the captured image, and the target cell region will be used as template image. The operator also inputs the position of the micropipette tip, which will initialize the automated injection progress (S2). Following initialization, a sample image is captured (S3). The sample image is preprocessed with a low-pass Gaussian filter (S4) and then converted into a binary image (S5). In addition, the edge information of the binary image is extracted using the Sobel edge detector (S6). The extracted contours are further smoothened through morphological operation (S7). The template image is processed using the same procedure (S4-S7), and the edge information of the template image is also obtained. The central position of the region on sampled image, where the edge information is similar to that of the template image, is located (S8). The correlation of edge information between the template image and each pattern region on the sampled image is calculated. It is determined whether the correlation is larger than a threshold (S9). If the condition in S9 is satisfied, the center of the matched pattern region in the sampled image is used to define the cell position. The position of the cell after injection will be removed from the system, and the target position of the cell with the smallest distance from the tip position will be chosen. If the condition in S9 cannot be satisfied or if all of the target cells are injected, the injection process stops. Once the position of the target cell is obtained, the target cell is aligned with the position of the micropipette tip in the x-axis by controlling the movement of the X-Y device carrier member (S10). The system will check whether alignment is achieved or not (S11). If alignment is achieved, the target cell is moved toward the micropipette tip by controlling the X-Y device carrier member (S12). A signal is sent to the microinjector, which will trigger the injection event (S13). During the injection event, a predefined pressure is applied at the end of the micropipette opposite to the tip for a predefined duration. A certain amount of substance in the micropipette will then be delivered into the cell. Once injection is completed, the target cell is moved back to its previous position that is, aligned with the tip of the injection needle. The system will inject all the cells trapped in the cell-trapping device by looping S3-S14.

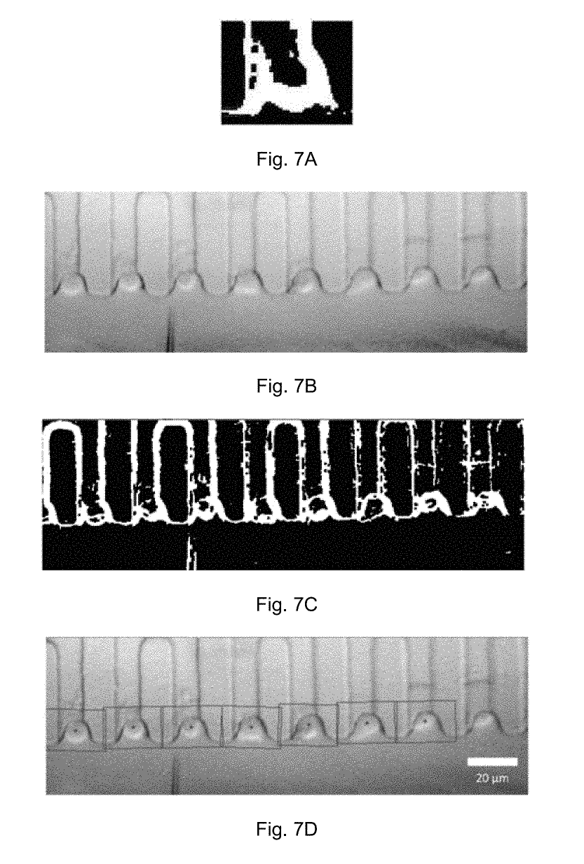

FIG. 7A to FIG. 7D further illustrates the image processing in embodiments of the present invention, wherein FIG. 7A refers to the template image provided, FIG. 7B shows an original (sample) image, FIG. 7C shows the reprocessed image, and FIG. 7D shows the recognition result after applying the edge template matching algorithm. By applying the above edge matching algorithm, the correlation between the actual (sample) image and the template image can be obtained. If the correlation is large enough, the center of the template is accepted as the position of the cell center (FIG. 7D). The performance of the image processing algorithm can be evaluated according to five criteria: true positive (TP), false positive (FP), true negative (TN), false negative (FN), and accuracy (ACC), which are defined as follows:

TP=no. of occupied cell-trapping microchannels recognized as targets

FP=no. of empty cell-trapping microchannels recognized as valid targets

TN=no. of empty cell-trapping microchannels ignored

FN =no. of occupied cell-trapping microchannels ignored

$$ACC = \frac{TP + TN}{no . of total cell \text{-} trapping microchannels}$$

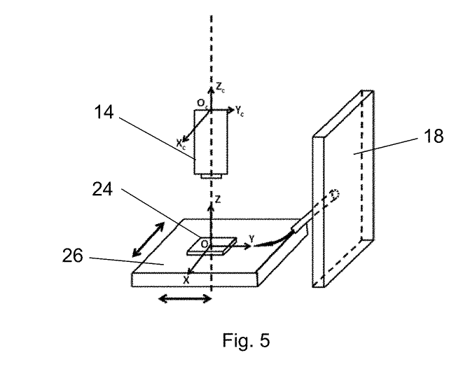

In an embodiment, a visual-guided position control scheme is applied in the method of the present invention. O_{c}-X_{c}Y_{c}Z_{c} is defined as the camera coordinate frame, where the origin O_{c} is defined at the top left corner of the image captured by the microscope as microscopic means. O-XYZ is defined as the cell coordinate frame, which is the same as that of an X-Y-Z positioning members. FIG. 5 shows the two coordinate frameworks. The relationship between the two coordinate frames is

$$[ \begin{matrix}X \\ Y \\ Z\end{matrix} ] = K [ \begin{matrix}{X}_{c} \\ {Y}_{c} \\ {Z}_{c}\end{matrix} ] + [ \begin{matrix}0 \\ 0 \\ {d}_{z}\end{matrix} ] \\ where \\ K = [ \begin{matrix}{k}_{x} & 0 & 0 \\ 0 & {k}_{y} & 0 \\ 0 & 0 & {k}_{z}\end{matrix} ] \qquad ( 1 )$$

represents a diagonal transformation matrix and d_{z} is the vertical distance between the origins of the vision detector like the camera frame and the coordinate frame.

The dynamics of a 3-DOF micromanipulation framework can be determined using Lagrange's equation of motion [8]

$$M [ \begin{matrix}\ddot{X} \\ \ddot{Y} \\ \ddot{Z}\end{matrix} ] + B [ \begin{matrix}\dot{X} \\ \dot{Y} \\ \dot{Z}\end{matrix} ] + [ \begin{matrix}0 \\ 0 \\ - {m}_{z} g\end{matrix} ] = [ \begin{matrix}{τ}_{x} \\ {τ}_{y} \\ {τ}_{z}\end{matrix} ] \\ where \\ M = [ \begin{matrix}{m}_{x} + {m}_{y} & 0 & 0 \\ 0 & {m}_{y} & 0 \\ 0 & 0 & {m}_{z}\end{matrix} ] \qquad ( 2 )$$

denotes the inertia matrix of the system; mₓ, m_{y}, m_{z} are the mass of the X-Y, and Z positioning members, respectively; B represents the effect of friction and system damping; −m_{z}g is the gravitational force;

[τₓτ_{y}τ_{z}]^{T}

is the input force to the X-Y-Z positioning elements. In this embodiment, dc-brushed motors are used to actuate the X-Y-Z positioning members. Given that the input forces are proportional to the currents to the motors,

[τₓτ_{y}τ_{z}]^{T}=[Kₘ_{ₓ}IₓKₘ_{ₓ}I_{y}Kₘ_{ₓ}I_{z}]^{T} (3)

Is obtained where Kₘₓ, K_{my}, and K_{mz} are constants which depend on the armature coil and magnetic flux density; Iₓ, I_{y}, and I_{z} are the currents flowing through the motors for the X-Y-Z positioning elements.

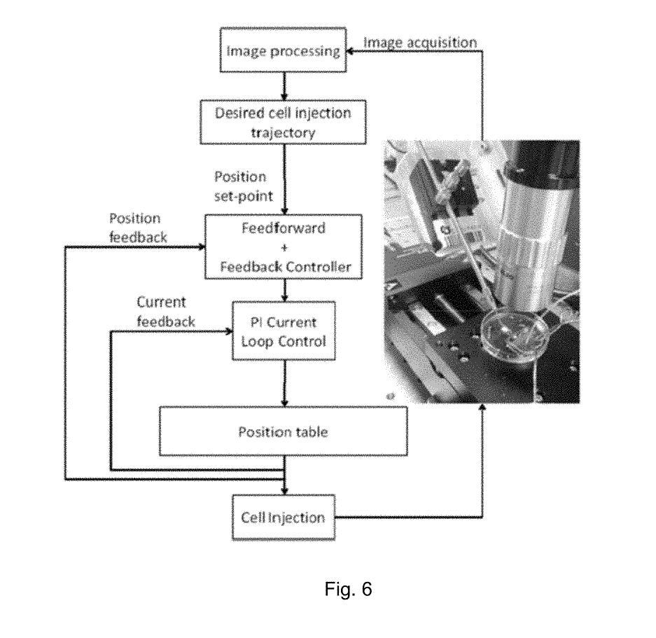

A visual-guided position control scheme, as shown in FIG. 6, is applied in embodiments to achieve automated cell injection. Initially, the injection needle such as the micropipette and the cell-trapping device are aligned with the Z-axis direction to stay in the focused plane of the CCD camera. After alignment, the positions of the micropipette and the cell-trapping device in the Z-axis remain unchanged throughout the injection process, and the automated injection is accomplished in the X-Y plane only. The control scheme includes the identification of cell position using image processing technology and the control of the X-Y device carrier member to drive the cell-trapping device to complete the injection process. Considering that the frame rate of the CCD camera is only 60 Hz, the use of visual feedback in the controller reduces the sampling frequency and degrades injection performance. To solve this problem, a motor encoder mounted on the X-Y device carrier member is used in embodiments to measure positions with high sampling frequency (for example 1000 Hz). Image acquired by the CCD camera is used to locate the cell and determine the destination of the injection motion. The information is then used for guiding the X-Y device carrier member to move toward the micropipette.

The control algorithm for each motion axis employs a simple feedforward plus PID feedback control in the form of

$$I = {K}_{p} {e}_{p} + {K}_{i} {\int }_{0}^{t} {e}_{p} ( τ ) dr + {K}_{d} \frac{{de}_{p} ( t )}{dt} + {K}_{f} {\ddot{x}}_{d} \qquad ( 4 )$$

where I denotes the current control input, eₚ is the position error, Kₚ, Kᵢ, and K_{d} are PID control gains, K_{f} is a feedforward control gain, and

\ddot{x}_{d}

is the desired acceleration set by the controller. The current control input I then goes to an inner current control loop, which is designed with a PI control scheme.

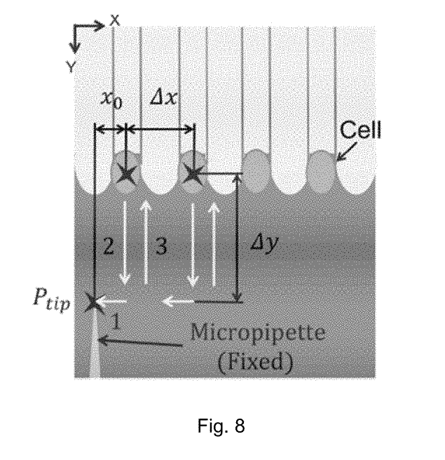

FIG. 8 illustrates an embodiment with an injection path plan that includes three paths for 1) aligning the cell with the micropipette (Path 1), 2) moving the cell toward the micropipette (Path 2), and 3) moving the cell back to its original position (Path 3). In this embodiment, the micropipette is fixed, and the cell located in one cell-trapping microchannel of the cell-trapping device moves straight toward the micropipette (for example, along the Y-axis direction) to complete one single-cell injection. The use of a straight-line path can minimize the damage to the cell during injection. Some sophisticated path planning algorithms (Wu, Y., IEEE/ASME Trans. Mechatronics, 2012, 18, 706-713, Wang, J., IEEE/ASME Trans. Mechatronics, 2013, 19, 549-558, Suzuki, H. and Minami, M., IEEE/ASME Trans. Mechatronics, 2005, 10, 352-357, Conticelli, F. and Allotta, B., IEEE/ASME Trans. Mechatronics, 2001, 6, 356-363) have been proposed, which may be used in embodiments for cell injection; however, given that the regular structure of the proposed cell-trapping device design can greatly simplify the process, the following simplified path plan can be applied in a preferred embodiment:

Define Δₓᵢ and Δ_{yi} as the horizontal and vertical distances between the micropipette tip (Pₜᵢₚ) and the i^{th} target cell, respectively. In the experiment, Δₓᵢ and Δ_{yi} vary among different cells, as shown in FIG. 7A to FIG. 7D. The travel distance of the cell-trapping device to complete one single-cell injection is:

d_{f}=Δxᵢ+2Δyᵢ.

To complete the injection of n cells, the total traveled distance of the cell-trapping device is

$${D}_{i} = {\sum }_{i = 6}^{N} Δ {x}_{i} + 2 Δ {y}_{i} .$$

Before injection, the tip position of the micropipette (Pₜᵢₚ) and the template image of the filled cell-trapping microchannels are determined. All the cell-trapping microchannels occupied by cells are checked to trigger injection. The first cell-trapping microchannel is aligned vertically with the micropipette, and a pulse is sent to the microinjector after the micropipette inserts the cell. The injection pressure and time are adjusted. The cell-trapping device is moved away from the micropipette, and the system starts to search for the second cell-trapping microchannel. The process repeats until all the cells in cell-trapping microchannels are injected.

Although the invention is described with reference to the specific embodiment described above, the invention is not intended to be limited to the above-mentioned details. Various modifications and improvements can be made according to certain applications without departing from the invention. The following non-limiting examples demonstrate the advantages of the invention.

### EXAMPLES

### Example 1

### Preparation of a Cell-Trapping Device of the Present Invention

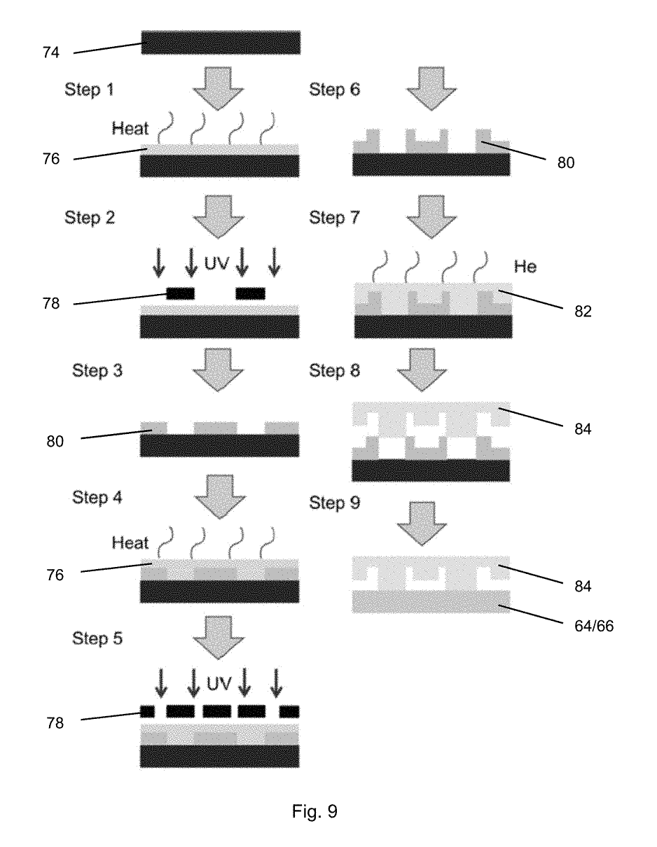

A cell-trapping device of the present invention was prepared by a soft lithography replica molding technology with PDMS (SYLGARD). The fabrication process is illustrated in FIG. 9. Prior to fabrication, two UV masks with features of two layers were printed on a transparency with a high-resolution printer. For the second layer, which had a thickness of 3-5 μm, mold fabrication was initiated by spin coating SU-8 negative photoresist (GM1050, Gersteltec Sarl) on a clean 3-in silicon wafer 74, where the thickness of SU-8 photoresist 76 was dependent on the size of the target cells. The diameter of the target cells was set to be 10-25 μm. Hence, 5-μm SU-8 was spin coated on the silicon wafer to fabricate the cell-trapping device. The SU-8 photoresist was then heated on a hotplate (AccuPlate, Labnet) (step 1). The temperature increased from room temperature to 95° C. in 5 min. It was baked at 95° C. for 3 min. After it was cooled to room temperature, it was covered with a first UV mask 78 and irradiated by UV light (365 nm) (step 2). After UV exposure, it was heated on the hotplate (from room temperature to 80° C. in 5 min) and then baked at 80° C. for 2 min. The exposed area of SU-8 photoresist formed crosslinks 80 during postexposure baking. After it was cooled to room temperature, the unexposed area of photoresist on was removed using the SU-8 developer (step 3).

The same procedures were used for the first layer (steps 4 to 6), which had a height of 10-15 μm. Before the second UV exposure, a second UV mask 78 was aligned precisely with the second layer using a mask aligner (MA6, Karl Suss).

The cell-trapping device was fabricated by replica molding with PDMS (SYLGARD 184, DowCorning) and the forming member (step 7). The PDMS was mixed with its curing agent in 10:1 (w/w) and poured on the forming member. The forming member with PDMS was degassed to remove air bubbles inside PDMS. The PDMS mixture 82 was cured by baking in an oven. The cured PDMS was peeled off from the forming member (step 8) and trimmed under a microscope with a 5×objective (Mitutoyo, Japan), which was followed by punching the outlet on the PDMS. The trimmed PDMS sample was cleaned and bonded on a glass surface as cover portion 64 or base portion 66 using the plasma bonding technique (step 9).

### Example 2

### Simulations with a Cell-Trapping Device of the Present Invention

Simulations were performed to test preliminary pressure setting parameters and to verify the effectiveness of the cell-trapping device. The finite-element analysis software, Comsol Multiphysics, was used for the simulation. The incompressible Naïve-Stoke equation was used to simulate the velocity and pressure distribution, in which the cell was assumed to be a perfect sphere with a diameter of 19 μm. In the simulation, the height and the width of the channel were set as 20 μm. The fluid flow was assumed to be laminar. The simulation was performed in two steps. In the first step, the three-dimensional structure of the whole cell-trapping device (see FIG. 2C) was modeled, which was used to determine the suitable pressure for trapping cells with minimal deformation while moving the cells into the traps at a reasonable speed. Note that the typical force required to deform a cell is in the order of 10 pN. Assuming that the fluid flow is laminar, the drag force acting on the cell can be estimated by Stokes' law, so the fluid velocity can be determined. The inlet fluid velocity was set to 50 μm/s to minimize the deformation of the trapped cell. The outlet was set as an open boundary. The difference between the outlet and the inlet pressures was estimated as −157.6 Pa in simulation. The actual outlet pressure applied in the experiment may be smaller such that the cell will not squeeze through the cell-trapping microchannels. In the second step, the cell-trapping microchannels of the cell-trapping device were modeled to verify the effectiveness of the cell-trapping device in trapping only one single cell at each cell-trapping microchannel. Two simulations have been performed to obtain the velocity distributions of the occupied and the empty cell-trapping microchannels, respectively, in which the inlet fluid velocity was set as 50 μm/s, and the outlet was set as open boundary.

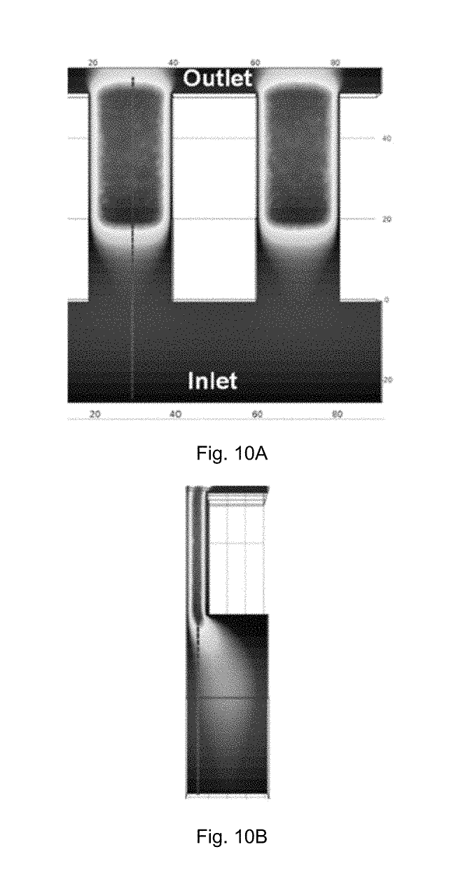

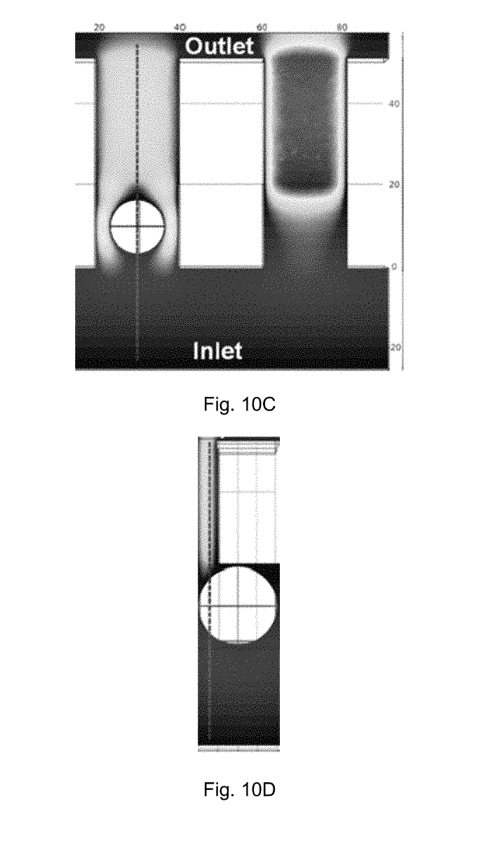

FIG. 10 shows the simulation results, where the slice plot represents the flowing velocity inside the cell-trapping channel.

When the cell-trapping microchannel is empty, flow velocity is high.

According to Stokes' law, the dragging force is proportional to the flow velocity; hence, the flow drags the cell to the cell receiving part. When the cell-trapping microchannel is occupied, flow velocity is low. The fluid velocity of the empty microchannels is about two times larger than that of the occupied microchannel. As a result, the flow redirects the incoming cells to other empty microchannels, preventing the occupied microchannel from being overloaded. The follow-on experiments have also verified that each cell-trapping microchannel traps only one single cell.

### Example 3

Assembly of an Apparatus of the Present Invention

An apparatus is provided comprising an X-Y device carrier member in form of a X-Y stage (PIM-L01, Physik Instrumente Co., Ltd.) , a needle holding member in form of a Z-axis linear table (KR30H06A, THK CO., LTD.), a micropipette (BF-100-50-15, Sutter Instrument), a cell-trapping device, and a pressure-based microinjector (IM-300, NARISHIGE). The motorized X-Y device carrier member has a resolution of 0.2 μm. The cell-trapping device is placed inside a petri dish, which is fixed in the X-Y device carrier member with two clamps. A glass micropipette, with an outer diameter of 1.0 mm and an inner diameter of 0.5 mm, is heated and pulled using a laser-based micropipette puller (P-2000, Sutter instrument). The micropipette is mounted on the Z needle holding member and connected to the microinjector via a pressure tube. The diameter of the micropipette tip is approximately 0.5 μm. The microinjector is connected to the outlet of the cell-trapping device and provides a negative pressure. The control unit consists of a computer and a motion controller (DCT0040, Dynacity Tech. Ltd.), with a sampling frequency of 4 kHz. The cell-detection unit consists of a CCD camera (STC-700, SENTECH) and a 20× objective (Mitutoyo, Kawasaki, Japan), which are mounted at the two ends of an observation tube (Infinity Tube, Boulder, Colo., USA). A light source (PL-800, Fiber-Lite) provides illumination. The image is captured using the PC2-Vision frame grabber (OC-PC2MVUM00, Dasal Corp.) and displayed with the image-processing library (Sapera Essential, Dasal Corp.). Both the injection module with the X-Y device carrier member, the cell-trapping device and the Z needle holding member with the micropipette and the cell-detection unit are placed inside a PMMA chamber, which is mounted on an anti-vibration table.

### Example 4

### Microinjection into HFF Cells

Human foreskin fibroblasts (HFF) were used in the cell injection experiment. The cells were maintained in Dulbecco's modified Eagle's medium (DMEM, Gibco) supplemented with 10% fetal bovine serum (FBS, Gibco), 100 U/mL penicillin, and 100 U/mL streptomycin in a humidified atmosphere of 37° C. and 5% CO₂. Before experiments, the cells were enzymatically detached from the cell culture plate and isolated to single cells. The cells were then suspended in the stated culture medium.

The cell-trapping device was sterilized by flowing 70% ethanol through the microchannels for 10 min. The microchannels were rinsed by flowing DMEM for 10 min. The cell-trapping device was then exposed to UV for 30 min and filled with the cell culture medium by connecting its outlet to a 1-mL syringe (BD Falcon). The cell-trapping device was degassed and fixed in a petri dish filled with the cell culture medium. The negative pressure was applied to the cell-trapping device by connecting the outlet to the microinjector through polyethylene tubing.

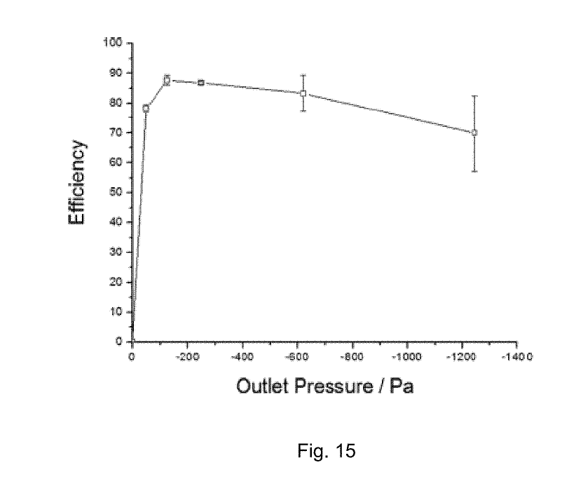

During experiments, the cells were trapped into cell-trapping microchannels of the cell-trapping device within 10 min upon their introduction. The cells that were not trapped were removed by gently flushing the cell culture medium near the cell-trapping area using a pipette. To easily verify the injection effect, tetramethylrhodamine isothiocyanate (TRITC) was injected into the cells. The micropipette was bent and backfilled with 1-mg/mL TRITC-Dextran before it was mounted on the Z needle holding table. The micropipette tip was carefully aligned and inserted into the cell-trapping device. About 20 μL of the cell solution (˜1000 cells/μL) was transferred into the petri dish using a pipette. The effect of the negative pressure on the trapping efficiency was determined for optimizing the cell-trapping performance, with data summarized in FIG. 15. The trapping efficiency of the cell-trapping device attained a maximum of 87% when the negative pressure was —124.6 Pa. As observed from the microscope, the trapped cells were largely deformed when the negative pressure was higher than 249 Pa. When all the channels were occupied by cells, the negative pressure was reduced to 24.9 Pa for holding the cell in the channel. The pressure was maintained by the microinjector throughout the whole injection process. Before the cell injection experiment, the ratio of pixel to actual length k was calibrated as 2.13 pixels/μm. The relationship between the encoder count and the micrometer was calibrated as 15 counts/μm. The maximum speed and acceleration of the X-Y device carrier member were 0.22 mm/s and 1.76 mm/s², respectively.

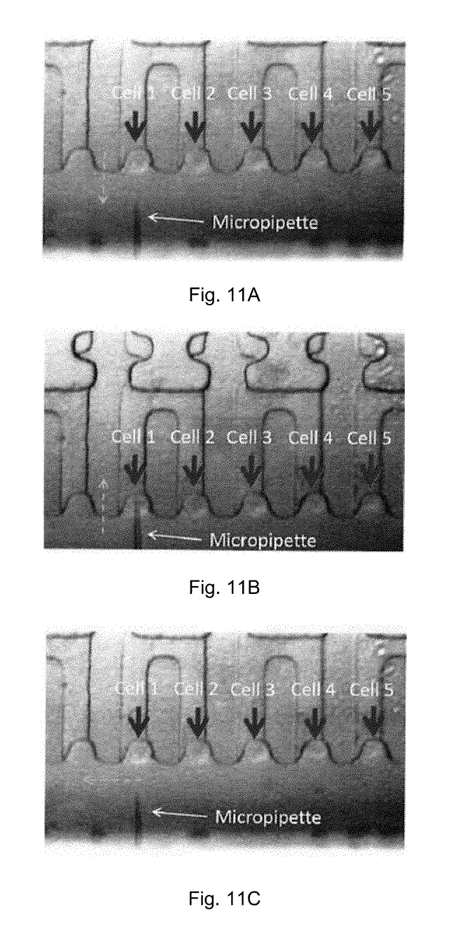

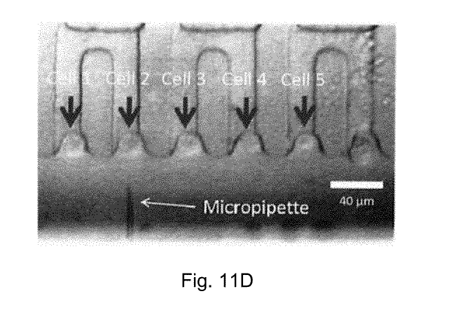

The cell was aligned with the micropipette (FIG. 11A to FIG. 11D). The cell-trapping device was moved toward the micropipette and then stopped for injection (FIG. 11B). After injection was completed, the cell-trapping device was moved back to the original position (FIG. 11C) and moved horizontally so that the next cell was aligned with the micropipette. The process was repeated until all the trapped cells were injected.

The total injection process for one single cell, including detecting the cell in the cell-trapping microchannel, moving the cell-trapping device on the device carrier member, and performing injection, took approximately 1.7 s. This finding was equivalent to an operation speed of 35.3 cells/min, which was much higher than other existing approaches (for example, 6 cells/min in Becattini, G. et al., IEEE J. Biomed. Health Informat., 2014, 18, 83-93).

To verify the cell recognition efficiency, a total of 377 cells were processed in the experiment. The cell recognition results are given in Table 1.

TABLE 1
Image processing results

| TP | TN | FP | FN | ACC (%) |
|---|---|---|---|---|
| 340 | 9 | 23 | 5 | 92.6 |

The accuracy of the cell recognition algorithm was 92.6%, indicating that the system can detect the occupied cell-trapping microchannels as targets and skip the empty cell-trapping microchannels efficiently. The system only incorrectly treated 7.4% of the examined cell-trapping microchannels. TN, FN, and FP are rare because most of the cell-trapping microchannels are occupied. In the above data set, the correlation threshold was set as 70%.

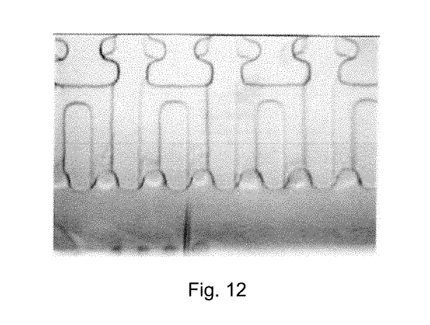

An image of the loaded cell-trapping device in the experiment is shown in FIG. 12. A summary of the trapping efficiency is given in Table 2.

TABLE 2
Cell-trapping results

| | | No. of | | |
|---|---|---|---|---|
| | Cell-trapping | cell-trapping | No. of | Trapping |
| Cell | microchannel | microchannels | trapped | efficiency |
| type | width (μm) | observed | cells | (%) |
| HFF | 20 | 735 | 608 | 82.7 |

The trapping efficiency is defined as the ratio of the number of the filled cell-trapping microchannels to the number of total cell-trapping microchannels. The measured efficiency was 82.7% (HFF) in the experiments. Notably, a high cell-trapping efficiency can help reduce FP in the recognition results. When most of the cell-trapping microchannels are occupied by cells, the chance of miscounting empty cell-trapping microchannels decreases.

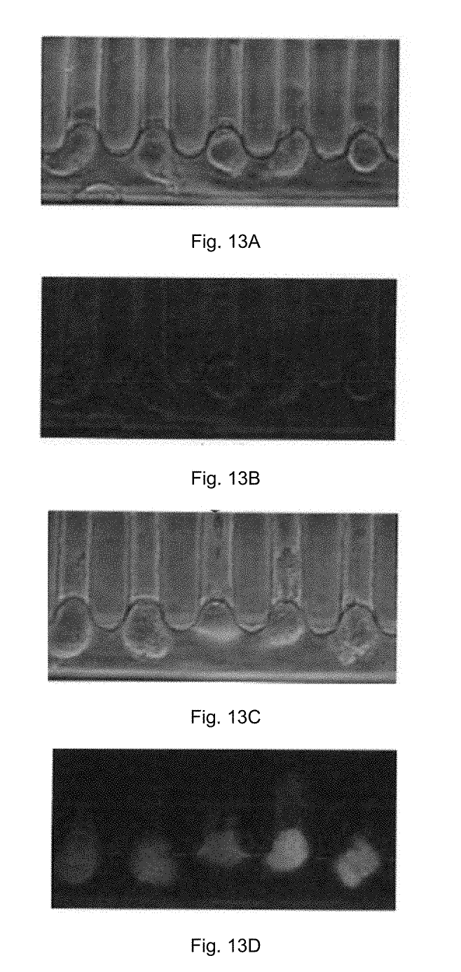

To further examine the injection effect, the injected cells were analyzed using a fluorescent microscope. HFFs with a diameter of 15 μm to 20 μm were applied and the height and width of the cell-receiving part of the cell-trapping microchannels were 15 μm and 20 μm, respectively. The loading cell concentration was ˜1000 cells per μL. The negative pressure applied to the cell-trapping device was 1.5 iH₂O (about 373 Pa), which generates a fluid flow that drags cells toward the cell holder outlet. The typical cell trapping time was 10 min. An injected cell should show red fluorescence if the dye is successfully injected into the cell. FIG. 13A to 13D show the images of HFF before and after injection of TRITC-Dextran.

FIG. 13A shows the bright field image of HFFs before injection, whereas FIG. 13B shows the fluorescent overlaid image of HFFs before injection. Before injection, no fluorescence signal was detected. FIG. 13C shows the bright field image of HFF after injection, whereas FIG. 13D shows the fluorescent overlaid image of HFF cells after injection. The overall injection efficiency was 88%, and the survival rate was 81.5%. The summary of the cell injection performance is given in Table 3.

TABLE 3
Cell-injection results

| | No. of | No. of | Injection |
|---|---|---|---|
| | injected | fluorescent | efficiency |
| Cell type | cells | cells | (%) |
| HFF | 657 | 581 | 88.4 |

The injection efficiency is defined as the ratio of the number of the fluorescent cells to the number of the total injected cells, namely

$$Injection efficiency = \frac{no . of fluorescent cells}{n o . of injected cells}$$

The overall injection efficiency was 88% for HFF, which value is better than that of manual injection performed by trained operators, which was about 40% as reported in Wang, W. et al.(Rev. Sci. Instrum., 2008, 79, 104302-1-104302-6). Furthermore, this result was better than two existing methods of flow constriction (Sharei, A. et al., Proc. Nat. Acad. Sci., 2013, 110, 2082-2087) and femtosecond laser delivery (Chakravarty, P. et al., Nature Nanotechnol., 2010, 5, 607-611), which has an efficiency of 70% (delivering 70-kDa dextran) and 35% (delivering FITC-BSA), respectively.

After cell injection, the injected cells were incubated for 24 h to examine the cell survival rate. The survival rate is defined as

$$Survival rate = \frac{no . of fluorescent cells after 24 h after injection}{no . of fluorescent cells right after injection}$$

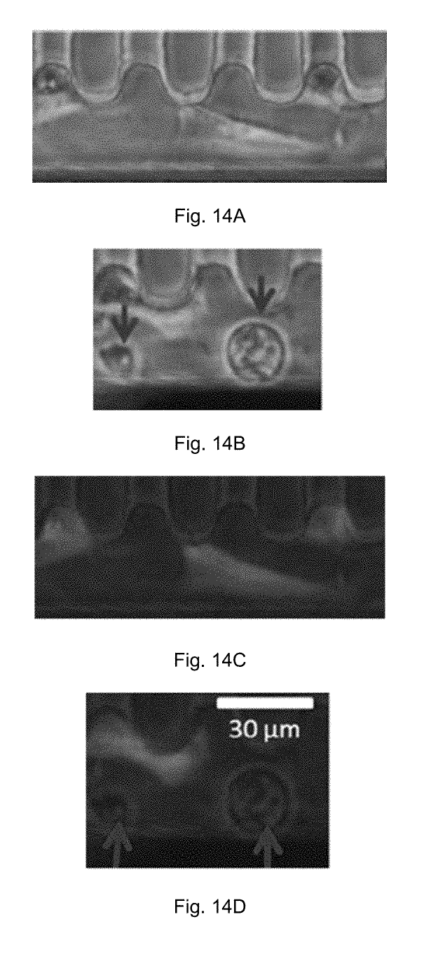

The survival rate for HFF was 81.5%. FIG. 14A to 14D show images of the cells at 24 h after injection. The survived cells exhibited normal morphology and were attached to the chip bottom, indicating that cell damage induced by injection was small. FIGS. 14B and 14D illustrate the dead cells, which became round and were detached from the device bottom.

### LIST OF REFERENCE SIGNS

10 Apparatus
12 Microinjector
14 Vision detector
16 Control unit
18 Needle holding member
20 Injection needle
22 Microscopic means
24 Cell-trapping device
26 Device carrier member
26a Device carrying surface
26b Surface opposite to device carrying surface
28 Anti-vibration means
30 Light source
32 Motion controller
34 X-Y plane
36 Cell receiving part
38 Fluid transfer part
40 Cell-trapping microchannels
42 Second layer
44 First layer
46 Microchannel portion
48 Height of microchannel in cell receiving part and the outlet microchannels
48a Height of microchannel in fluid transfer part
50 Width of microchannel
52 Outlet
54 Cell-trapping area
56 Outlet microchannels
58 Outlet area
60 Inlet area
62 Inlet
64 Cover portion
66 Base portion
68 Bent micropipette
70 Cell
72 Liquid transfer pipette
74 Silicon wafer
76 SU-8 photoresist
78 UV mask
80 Cross-linked photoresist
82 Mixture of PDMS with curing agent
84 Cured PDMS

1. A cell-trapping device for trapping a plurality of cells with an average diameter of at most 25 μm for high-throughput microinjection of an injectant into the cells,
said cell-trapping device comprising a microchannel portion having formed therein a cell-trapping area comprising a plurality of cell-trapping microchannels configured to trap one cell per cell-trapping microchannel.

2. The cell-trapping device of claim 1, wherein the cell-trapping area comprises more than 200 cell-trapping microchannels.

3. The cell-trapping device of claim 1, wherein the cell-trapping microchannels are arranged substantially in parallel at regular intervals in a row along a linear axis through the cell-trapping device.

4. The cell-trapping device of claim 1, wherein the microchannel portion further comprises an inlet area having an inlet constructed for receiving the plurality of cells in a fluid and an outlet area having outlet microchannels for directing the fluid along with untrapped cells smaller than the cell-trapping microchannels to an outlet for releasing the fluid along with the untrapped cells, wherein the cell-trapping area is arranged between outlet area and inlet area.

5. The cell-trapping device of claim 4, wherein the cell-trapping microchannels of the microchannel portion proceed into the outlet microchannels of the outlet portion and the microchannel portion is formed by a first layer with a height of up to about 20 μm arranged on a second layer with a height of up to about 5 μm and wherein the first layer and the second layer comprise polydimethylsiloxane.

6. The cell-trapping device of claim 5, wherein the cell-trapping microchannels and the outlet microchannels are formed by recesses in the first layer and/or in the second layer, which recesses are formed substantially perpendicular to the horizontal dimensions of the first layer and second layer and proceed substantially parallel to both horizontal dimensions of the first layer and/or the second layer.

7. The cell-trapping device of claim 6, wherein the recesses forming the outlet microchannels have a height between about 0.8 and about 1×the average cell diameter and a width of at least about 1×the average cell diameter, and wherein the cell-trapping microchannels have a cell receiving part formed by recesses with a height and a width of between about 0.8 and about 1×the average cell diameter and a fluid transfer part formed by recesses with a width of between about 0.8 and about 1×the average cell diameter and a high of at most about 0.5×the average cell diameter.

8. The cell-trapping device of claim 7, wherein the fluid transfer part is formed by recesses with a height of at most about 0.25×the average cell diameter and wherein the cell-trapping device further comprises at least one of a cover portion or a base portion with cover portion and base portion comprising glass.

9. The cell-trapping device of claim 1 which is transparent for visible light and which comprises at least 356 cell-trapping microchannels in the cell-trapping area.

10. An apparatus for high-throughput microinjection of an injectant into a plurality of cells with an average diameter of at most 25 μm comprising:
a cell-trapping device as claimed in claim 1; and
an injection needle with a tip arranged to be stuck into the cells trapped in the cell-trapping area of the cell-trapping device to inject the injectant into the trapped cells.

11. The apparatus of claim 10 for high-throughput microinjection with a throughput of at least about 30 cells/min into more than 100 cells, the cells consisting of human cells having an average diameter of less than about 25 μm and wherein the injectant is selected from at least one of DNA, RNA, polypeptides or proteins.

12. The apparatus of claim 10 further comprising:
a device carrier member for carrying the cell-trapping device;
a needle holding member for supporting the injection needle;
a control unit for guiding the injection needle to the trapped cells;
a cell-detection unit to detect the trapped cells and to generate a signal for initiating the microinjection;
a pressure-based microinjector; and
anti-vibration means.

13. The apparatus of claim 12, wherein the device carrier member has a device carrying surface facing towards the cell-trapping device which is in a horizontal position substantially parallel to an X-Y plane which is parallel to level ground, and wherein the device carrier member is arranged such that it can move the cell-trapping device at least along a X direction and along an Y direction perpendicular to the X direction in the X-Y plane.

14. The apparatus of claim 13, wherein the injection needle is mounted on the needle holding member on a surface of the needle holding member which is arranged substantially perpendicular to the X-Y plane.

15. The apparatus of claim 14 comprising:
a control unit comprising a computer and a motion controller for controlling the position of the device carrier member in the X-Y plane and/or of at least a portion of the needle holding member in a Z direction perpendicular to the X-Y plane; and
a cell detection unit comprising a vision detector, microscopic means and a light source providing illumination to the microscopic means, which cell-detection unit is arranged on top of the cell-trapping device facing towards the surface of the cell-trapping device which is opposite to the surface of the cell-trapping device facing towards the device carrying surface of the device carrier member; and
an anti-vibration member onto which the device carrier member with the surface opposite to the device carrying surface and the needle supporting member are placed.

16. The apparatus of claim 12, wherein the microinjector is connected to the cell-trapping device and the injection needle and provides negative pressure to the cell-trapping device for trapping the cell, and positive pressure to the injection needle.

17. A method for microinjection of an injectant into a plurality of cells having an average diameter of at most 25 μm comprising steps of:
(i) providing an apparatus as claimed in claim 10;
(ii) introducing a plurality of cells into the cell-trapping device;
(iii) trapping the cells in the cell-trapping microchannels in the cell-trapping area in the microchannel portion of the cell-trapping device such that a cell-trapping microchannel traps one cell;
(iv) inserting an injection needle with the tip into the cell-trapping area in the microchannel portion of the cell-trapping device and injecting the injectant subsequently into a plurality of trapped cells.

18. The method of claim 17, wherein the microchannel portion of the cell-trapping device further comprises an inlet area having an inlet constructed for receiving the plurality of cells in a fluid and an outlet area having outlet microchannels for directing the fluid along with untrapped cells smaller than the cell-trapping microchannels to an outlet for releasing the fluid along with the untrapped cells, wherein the cell-trapping area is arranged between outlet area and inlet area; and wherein the cell-trapping device comprises at least 200 cell-trapping microchannels in the cell-trapping area; and wherein
step (ii) comprises applying the cells in the fluid to the inlet of the cell-trapping device;
step (iii) comprises applying a negative pressure of about 124.6 Pa to less than about 400 Pa at the outlet of the cell-trapping device for cell trapping in the cell-trapping microchannels in the cell-trapping area; and wherein
inserting the injection needle into the cell-trapping area in step (iv) includes bending the injection needle while inserting the tip into the cell-trapping area of the cell-trapping device for obtaining a needle tilt angle of more than 70°.

19. The method of claim 17, wherein the provided apparatus further comprises a device carrier member for carrying the cell-trapping device;
a needle holding member for supporting the injection needle;
and wherein step (iv) comprises steps of:
(a) inserting the injection needle with the tip into the cell-trapping area in the microchannel portion of the cell-trapping device;
(b) aligning a first trapped cell with the tip and moving the cell-trapping device in the direction of the trapped cell by moving the cell-trapping device in a direction to the tip and perpendicular to said direction such that the tip is stuck into the first trapped cell;
(c) injecting the injectant into the first trapped cell;
(d) moving the cell-trapping device away from the tip and to a second trapped cell by moving the cell-trapping device in a direction opposite to the tip and subsequently perpendicular to said direction such that the tip is in front of the second trapped cell, (d) aligning the second trapped cell with the tip and moving the cell-trapping device in the direction of the second trapped cell by moving the cell-trapping device in a direction to the tip and perpendicular to said direction such that the tip is stuck into the second trapped cell;
(e) injecting the injectant into said second trapped cell;
and repeating steps (d) to (e) with a third and any further trapped cells until all trapped cells have received the injectant.

20. The method of claim 17, wherein step (iv) further comprises steps of:
searching the position of an uninjected trapped cell by calculating the correlation of edge information between a template image and each pattern region on the sample image;
determining whether the correlation is larger than a set threshold; and
if this condition is met, proceeding with the injection of the injectant into the cell.
